(19) United States
(12) Patent Application Publication
(10) Pub. No.: US 2026/0234199 A1
(43) Pub. Date: Aug. 13, 2026

(54) INHIBITORS OF IL11RA BASED SIGNALLING

(71) Applicant: Novo Nordisk A/S, Bagsvaerd (DK)

(72) Inventors: Jendrik Schoeppe, Copenhagen S (DK); Frantisek Hubalek, Herlev (DK); Inger Lautrup-Larsen, Virum (DK)

(21) Appl. No.: 19/532,299

(22) Filed: Feb. 6, 2026

(30) Foreign Application Priority Data
Feb. 7, 2025 (EP) 25156425.8

(51) Int. Cl. C07K 14/54; A61K 38/00

(52) U.S. Cl. CPC C07K 14/5431; A61K 38/00

(57) ABSTRACT

The present invention relates to novel IL11RA binding polypeptides and inhibitors and use thereof in medicine and in particular for inhibiting hIL11RA-induced signalling in patients in need thereof, such as patients suffering from metabolic-dysfunction associated steatohepatitis (MASH), cardiovascular disease (CVD), fibro-inflammatory disease and fibrotic diseases as well as pharmaceutical compositions and kits comprising such compounds.

### TECHNICAL FIELD

The present invention relates to compounds capable of binding to IL11RA and inhibiting IL11RA-based signalling and their use in medicine.

### INCORPORATION-BY-REFERENCE OF THE SEQUENCE LISTING

The instant application contains a Sequence Listing which has been submitted in XML format via the USPTO patent electronic filing system and is hereby incorporated by reference in its entirety. Said XML file, created on Feb. 2, 2026, is named 240032US01_Sequence_Listing.xml and is 46,166 bytes in size.

### BACKGROUND

Interleukin-11 (IL11)-mediated cell activation has been identified as a central mechanism for promoting fibrosis downstream of transforming growth factor beta 1 (TGFb1). The circulating cytokine IL11 engages its cognate receptor and mediates downstream cellular signalling through two mechanisms:
1) classical signalling: IL11 binds to the extracellular domain (ECD) of the transmembrane Interleukin-11 receptor subunit alpha (IL11RA) which in turn allows for interaction with the transmembrane co-receptor Interleukin-6 receptor subunit beta (IL6STP1, also known as gp130). Such a heterotrimeric complex (IL11:IL11RA:gp130) can then dimerize to form the functional hexameric signalling complex that activates different intracellular signalling profiles.
2) trans signalling: while the in vivo relevance of this pathway and its clinical significance are unproven it has been demonstrated that when IL11 binds to the soluble ECD of IL11RA (generated through cleavage from the cellular surface by metalloproteases such as ADAM10) the complex can engage membrane-bound gp130 and thereby induce signalling on cells which only express gp130 but not IL11RA.

While IL11 is minimally expressed in healthy tissue it has been shown that IL11 levels are upregulated in patients with fibrotic diseases. In addition, the fibrogenic potential of IL11 was demonstrated in patients treated with recombinant IL11 to treat thrombocytopenia during chemotherapy as it led to fibrosis induction resulting in its voluntary withdrawal from market. Based on these findings a growing body of literature has suggested IL11-induced signalling to be a master driver of fibrosis across tissues. Indeed, the current consensus supports the profibrotic capabilities of IL11 in many tissues including heart, lung, intestine, kidney, and liver as elevated IL11 levels have been demonstrated in patients and in precision-cut tissue slices across species. Blocking the IL11 pathway led to reduction of hepatocyte death and liver fibrosis, inflammation, and steatosis in mouse models of metabolic dysfunction-associated steatohepatitis (MASH) formerly known as non-alcoholic steatohepatitis (NASH). Hepatocytes highly express IL11RA and they secrete IL11 in response to lipid loading. Autocrine IL11 activity causes hepatocyte death through NOX4-derived ROS, activation of ERK, JNK and caspase-3, impaired mitochondrial function and reduced fatty acid oxidation. On the other hand, paracrine IL11 activity stimulates hepatic stellate cells and causes fibrosis. Interestingly, hepatocyte-specific deletion of IL11RA in MASH mice protects against liver steatosis, fibrosis and inflammation while reducing serum glucose, cholesterol and triglyceride levels and limiting obesity.

Furthermore, assessment of human genetic data analysis implicates IL11 signalling in cardiometabolic disease and confirmed elevated IL11 in plasma, kidney, heart and liver in fibrotic diseases. Elevated IL11 protein and IL11RA mRNA levels were detected in liver biopsies in MASH patients with advanced fibrosis.

Cardiovascular disease (CVD) represents a primary cause of death worldwide, with myocardial damage resulting eventually in cardiovascular fibrosis. Analysis shows high expression of IL11RA on cardiac fibroblasts which, when stimulated with IL11, promote fibrosis by increasing extracellular matrix production, cell motility, contraction, and invasion. The profibrotic nature of IL11 in CVD was accidently discovered as an adverse event in chemotherapy-induced thrombocytopenia patients who received IL11 drug Neumega resulting in atrial fibrosis in 15% of treated individuals. In addition, elevated IL11 has been associated with cardiac events in patients with chronic heart failure. In vitro studies have shown that IL11 is the most upregulated gene when human atrial fibroblasts were stimulated with profibrotic inducer TGFB1, and IL11 expression was highly correlated with the fibrotic response. Further support for IL11 roles in CVD came from preclinical studies showing that recombinant murine IL11 is profibrotic and required for mouse fibroblast activation and that germline deletion of IL11RA protects mice from cardiac and renal fibrosis while preserving organ function.

The known signalling mechanisms offer three possible options to specifically inhibit IL11-mediated signalling without interfering with the ubiquitous gp130 signalling:
1) Creating a compound the specifically binds to IL11 and competitively blocks IL11 from interacting with IL11RA
2) Creating a compound that specifically binds to IL11RA and inhibits the interaction with IL11
3) Creating a compound that binds to IL11RA but does not interfere with the IL11:IL11RA interaction but blocks formation of the heterotrimeric IL11:IL11RA:gp130 complex

See also: Schafer, S., et al., IL-11 is a crucial determinant of cardiovascular fibrosis. Nature 552, 110-115 (2017); Dong, J. et al. Hepatocyte-specific IL11 cis-signalling drives lipotoxicity and underlies the transition from NAFLD to NASH. Nat Commun 12, 66 (2021); Benjamin Ng et al., Interleukin-11 is a therapeutic target in idiopathic pulmonary fibrosis. Sci. Transl. Med. 11 (2019). Cook, S., Schafer, S., Hiding in plain sight: Interleukin-11 emerges as a master regulator of fibrosis, tissue integrity and stromal inflammation. Annu Rev Med. 2020 Jan. 27:71:263-276.

Antibodies that work through either of these three mechanisms have been developed and have been shown to be capable of inhibiting IL11-mediated signalling. Specifically, IL11-signalling inhibitors under development include antibodies that bind to IL11RA (see, for example, U.S. Pat. Nos. 9,796,782; and 9,340,618; and WO 2023/034809). However, there is a need in the art for improved compounds capable of inhibiting IL11-mediated signalling.

### SUMMARY

The present invention relates to compounds that are capable of binding to Interleukin-11 receptor subunit alpha (IL11RA), in particular human Interleukin-11 receptor subunit alpha (hIL11RA) and compounds capable of inhibiting Interleukin-11 (IL11)-mediated signalling, and use of such compounds in medicine as well as in pharmaceutical compositions and kits comprising such compounds.

In one aspect the invention relates to an IL11RA binding polypeptide capable of binding an epitope comprising the amino acid residues Y125, K150, L154, A156, F187, W188, S189, Q213, Q249, H251, F252, L253, D297, F298, L299 and D300 on hIL11RA according to SEQ ID NO: 1 as determined at 3.5 Å.

In one aspect the invention relates to an IL11RA binding polypeptide comprising the amino acid residue sequence selected from

i)
A₁S₂A₃T₄F₅P₆X₇Q₈C₉X₁₀X₁₁Q₁₂L₁₃M₁₄D₁₅L₁₆G₁₇F₁₈P₁₉X₂₀Y₂₁A₂₂V₂₃X₂₄A₂₅A₂6L₂₇X₂₈
Y₂₉T₃₀N₃₁G₃₂N₃₃C₃₄E₃₅X₃₆A₃₇A₃₈S₃₉L₄₀L₄₁F₄₂X₄₃R₄₄ according to SEQ ID NO: 2

wherein, independently of each other,
X₇ is R, V or I;
X₁₀ is A, L, D, E, T, V, I or Y;
X₁₁ is W, A, R or F;
X₂₀ is Q, A, R, V, L or F;
X₂₄ is I, A, H, V, L, F or Y;
X₂₈ is R, H, V, L, I, F or Y;
X₃₆ is R, A, H, T, Q, V, L, I, F or Y;
X₄₃ is A or H;
and
ii) an amino acid residue sequence having at least 90%, at least 91%, at least 92%, at least 93%, at least 94%, at least 95%, at least 96%, at least 97%, at least 98% or at least 99% identity to the sequence defined in i),

In one embodiment said polypeptide is an IL11RA inhibitor, such as a hIL11RA inhibitor.

In one aspect the invention relates to an IL11RA binding polypeptide capable of binding an epitope comprising the amino acid residues P141, R143, L145, P167, W168, P169, L175, V198, N199, P200 and A203 on hIL11RA according to SEQ ID NO:1 as determined at 3.5 Å.

In one aspect the invention relates to an IL11RA binding polypeptide comprising a binding motif (BM), said (BM) consisting of an amino acid residue sequence selected from

iii)
XₐWX_{b}AWX_{c}EIX_{d}XₑLPNLNX₁WQX_{g}AAFIXₕSLXᵢ according to SEQ ID NO: 3

wherein, independently of each other,
Xₐ is A or L;
X_{b} is Q or A;
X_{c} is D or A;
X_{d} is D, A, T, V, I or L;
Xₑ is H, A, R, S, T, Q, I, L, F or Y;
X_{f} is P or A;
X_{g} is K, A, T or I;
Xₙ is L or A;
Xᵢ is L or A;
and
iv) an amino acid residue sequence which has at least 90%, at least 91%, at least 92%, at least 93%, at least 94%, at least 95%, at least 96%, at least 97%, at least 98% or at least 99% identity to the sequence defined in iii).

In one embodiment said polypeptide comprises an amino acid residue sequence selected from

v)
V₁D₂N₃R₄F₅N₆X₇E₈-(BM)-
X₃₆D₃₇P₃₈S₃₉Q₄₀W₄₁A₄₂N₄₃L₄₄L₄₅X₄₆E₄₇A₄₈R₄₉R₅₀L₅₁N₅₂D₅₃A₅₄Q₅₅A₅₆P₅₇X₅₈ according
to SEQ ID NO: 4

wherein, independently of each other,
X₇ is R or E;
X₃₆ is D or E;
X₄₆ is A or E;
X₅₈ is R or E;
and
vi) an amino acid residue sequence having at least 90%, at least 91%, at least 92%, at least 93%, at least 94%, at least 95%, at least 96%, at least 97%, at least 98% or at least 99% identity to the sequence defined in v).

In a preferred embodiment the IL11RA binding polypeptide is a hIL11RA binding polypeptide capable of binding to hIL11RA according to SEQ ID NO:1.

In one aspect the invention relates to an IL11RA inhibitor comprising a first IL11RA binding polypeptide capable of binding an epitope comprising the amino acid residues Y125, K150, L154, A156, F187, W188, S189, Q213, Q249, H251, F252, L253, D297, F298, L299 and D300 on hIL11RA according to SEQ ID NO:1 as determined at 3.5 Å, and a second IL11RA binding polypeptide capable of binding an epitope comprising the amino acid residues P141, R143, L145, P167, W168, P169, L175, V198, N199, P200 and A203 on hIL11RA according to SEQ ID NO:1 as determined at 3.5 Å.

In one aspect the invention relates to an IL11RA inhibitor comprising a first IL11RA binding polypeptide comprising the amino acid residue sequence selected from

vii)
A₁S₂A₃T₄F₅P₆X₇Q₈C₉X₁₀X₁₁Q₁₂L₁₃M₁₄D₁₅L₁₆G₁₇F₁₈P₁₉X₂₀Y₂₁A₂₂V₂₃X₂₄A₂₅A₂₆L₂₇X₂₈Y₂₉
T₃₀N₃₁G₃₂N₃₃C₃₄E₃₅X₃₆A₃₇A₃₈S₃₉L₄₀L₄₁F₄₂X₄₃R₄₄ according to SEQ ID NO: 2

wherein, independently of each other,
X₇ is R, V or I;
X₁₀ is A, L, D, E, T, V, I or Y;
X₁₁ is W, A, R or F;
X₂₀ is Q, A, R, V, L or F;
X₂₄ is I, A, H, V, L, F or Y;
X₂₈ is R, H, V, L, I, F or Y;
X₃₆ is R, A, H, T, Q, V, L, I, F or Y;
X₄₃ is A or H;
and
viii) an amino acid residue sequence having at least 90%, at least 91%, at least 92%, at least 93%, at least 94%, at least 95%, at least 96%, at least 97%, at least 98% or at least 99% identity to the sequence defined in vii);
and
a second IL11RA binding polypeptide comprises a binding motif (BM) which consists of an amino acid residue sequence selected from

ix)
XₐWX_{b}AWX_{c}EIX_{d}XₑLPNLNX₁WQX_{g}AAFIXₕSLXᵢ according to SEQ ID NO: 3

wherein, independently of each other,
Xₐ is A or L;
X_{b} is Q or A;
X_{c} is D or A;
X_{d} is D, A, T, V, I or L;
Xₑ is H, A, R, S, T, Q, I, L, F or Y;
X_{f} is P or A;
X_{g} is K, A, T or I;
Xₕ is L or A;
Xᵢ is L or A;
and
x) an amino acid residue sequence which has at least 90%, at least 91%, at least 92%, at least 93%, at least 94%, at least 95%, at least 96%, at least 97%, at least 98% or at least 99% identity to the sequence defined in ix).

In one such embodiment the second IL11RA binding polypeptide comprises the amino acid residue sequence selected from

xi)
V₁D₂N₃R₄F₅N₆X₇E₈-(BM)-
X₃₆D₃₇P₃₈S₃₉Q₄₀W₄₁A₄₂N₄₃L₄₄L₄₅X₄₆E₄₇A₄₈R₄₉R₅₀L₅₁N₅₂D₅₃A₅₄Q₅₅A₅₆P₅₇X₅₈ according
to SEQ ID NO: 4

wherein, independently of each other,
X₇ is R or E;
X₃₆ is D or E;
X₄₆ is A or E;
X₅₈ is R or E;
and
xii) an amino acid residue sequence having at least 90%, at least 91%, at least 92%, at least 93%, at least 94%, at least 95%, at least 96%, at least 97%, at least 98% or at least 99% identity to the sequence defined in xi).

In a further aspect the present invention relates to IL11RA inhibitors comprising a half-life extending moiety allowing for extended plasma half-life, such as an albumin-binding domain (ABD).

In one such embodiment the present invention relates to IL11RA inhibitors such as Chem. 10, Chem. 11, Chem. 12, Chem. 13 and Chem. 14 as disclosed herein.

In a further aspect the present invention relates to methods of producing the compounds as disclosed herein.

In a further aspect the present invention relates to pharmaceutical compositions comprising such compounds and medicinal use of such compounds and compositions for the treatment of metabolic-dysfunction associated steatohepatitis (MASH), cardiovascular disease (CVD), a fibro-inflammatory disease or fibrotic diseases in patients in need thereof.

In a further aspect the present invention relates to a kit comprising (i) a compound as disclosed herein, such as an IL11RA inhibitor, and (ii) instructions for use.

### BRIEF DESCRIPTION OF FIGURES

FIG. 1 shows a non-limiting graphical illustration of the modular composition of IL11RA inhibitor compounds of the present invention comprising a half-life extending moiety (left), and an IL11RA inhibitor comprising two IL11RA binding polypeptides (right).

FIG. 2 shows in silico predicted binding sites (epitope “I” and “II”) of two different IL11RA binding polypeptides (light grey cartoon) on the extracellular domain (ECD) of IL11RA (dark cartoon).

FIGS. 3A and B shows SPR sensorgrams of competition binding experiments. The different injections are indicated by “Injection A” and “Injection B” in the sensorgrams.

FIG. 4 shows the acute induction of liver pSTAT3 levels in B-hIL11RA mice 30 minutes after administration of recombinant murine IL11 (rmIL11) and the inhibition of rmIL11-induced liver pSTAT3 by Chem. 13 administered 30 minutes prior to rmIL11 at 3 different doses. ***p<0.001: Compared to Vehicle 1+rmIL11 treated B-hIL11RA mice.

### BRIEF DESCRIPTION OF SEQUENCES

SEQ ID NO:1 represents the amino acid residue sequence of hIL11RA.

SEQ ID NO:2 represents the amino acid residue sequence of a first group of IL11RA binding polypeptides with fixed and non-fixed residues.

SEQ ID NO:3 represents the amino acid residue sequence of the binding motif (BM) of a second group of IL11RA binding polypeptides with fixed and non-fixed residues.

SEQ ID NO:4 represents the amino acid residue sequence of a second group of IL11RA binding polypeptides, including the (BM) of SEQ ID NO:3, with fixed and non-fixed residues.

SEQ ID NOs: 5 and 6 represent the amino acid residue sequence of IL11RA binding polypeptides from a first group.

SEQ ID NOs: 7-11 represent the amino acid residue sequence of IL11RA binding polypeptides from a second group.

SEQ ID NO:12 represents the amino acid residue sequence of an albumin-binding domain.

SEQ ID NO:13 represents the amino acid residue sequence of a (GAQP) 4-repeat linker.

SEQ ID NO:14-16 represent the amino acid residue sequences of further linkers.

SEQ ID NOs: 17-21 represent the amino acid residue sequences of IL11RA inhibitors.

SEQ ID NOs: 22 and 23 represent the amino acid residue sequences of IL11RA binding polypeptides as used in Example 9 herein.

SEQ ID NOs: 24-27 represent the amino acid residue sequences of IL11RA inhibitors as used in Example 3 herein.

SEQ ID NOs: 28-30 represent the amino acid residue sequences of initial IL11RA binding polypeptides as identified in Example 1 herein.

SEQ ID NO:31 represents the amino acid residue sequence of the (BM) of Chem. 12.

SEQ ID NO:32 represents the amino acid residue sequence of a biotinylated hIL11RA construct used in Examples 6, 9, 10 and 11 herein.

SEQ ID NO:33 represents the amino acid residue sequence of an IL11RA binding polypeptide as used in Example 9 herein.

SEQ ID NO:34 represents the amino acid residue sequence of hIL11 as used in Example 9 herein.

SEQ ID NO:35 represents the amino acid residue sequence of an IL11RA binding polypeptide as used in Example 10 herein.

### DESCRIPTION

The present invention relates to compounds that are capable of binding to Interleukin-11 receptor subunit alpha (IL11RA), such as human IL11RA (hIL11RA), and compounds capable of inhibiting Interleukin-11 (IL11)-mediated signalling, and use thereof in medicine as well as in pharmaceutical compositions and kits comprising such compounds.

Increased hIL11 levels are thought to drive progression of fibrosis in diverse fibro-inflammatory disease or fibrotic diseases. Antibodies that inhibit IL11-mediated signalling through binding to the soluble cytokine IL11 or the ECD of IL11RA thereby preventing formation of the heterotrimeric signalling complex composed of IL11:IL11RA:gp130 are known in the art.

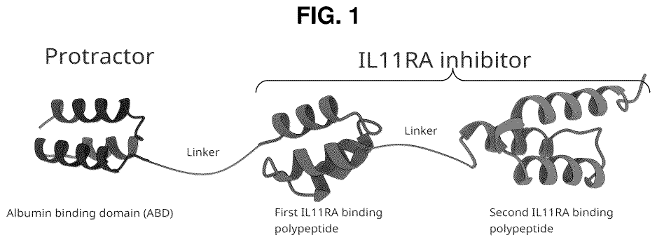

The present inventors have developed novel, non-antibody-based IL11RA inhibitors—in some instances comprising a half-life extending moiety (protractor)—that simultaneously bind two non-overlapping epitopes on IL11RA with high affinities and show potent IL11-mediated signalling blockade, as well as (intermediate) IL11RA binding polypeptides serving as components/modules in such inhibitors. See FIG. 1 and Example 4 herein.

### Definitions

In order that the present invention may be more readily understood, certain terms are first defined.

An asterisk (*) in a chemical formula designates a point of attachment.

The term “a” or “an” is intended to mean “one or more.” The term “comprise” and variations thereof such as “comprises” and “comprising,” when preceding the recitation of a step or an element, are intended to mean that the addition of further steps or elements is optional and not excluded.

The term “amino acids” or “amino acid residues” includes canonical amino acids (which are genetically encoded), and unnatural amino acids. Non-limiting examples of unnatural amino acids are Aib (α-aminoisobutyric acid), desamino histidine (alternative name 3-(imidazol-4-yl) propanoic acid, abbreviated Imp (imidazopropionyl) and the d-isomers of the canonical amino acids. All amino acid residues within the polypeptide for which the optical isomer is not stated is herein to be understood to mean the L-isomer, unless otherwise specified.

The term “avidity” as used herein refers to the overall strength of the interaction between a multivalent ligand and its corresponding target. Unlike affinity, which describes the strength of a single binding site interaction, avidity encompasses the cumulative binding strength resulting from multiple interactions. Avidity is influenced by both the individual affinities of the binding sites and the spatial arrangement and flexibility of the interacting components. High avidity can result in a more stable and longer-lasting interaction, even if the individual binding affinities are relatively weak.

The term “binding affinity” is herein used as a measure of the strength of a non-covalent interaction between two molecules, e.g. a polypeptide and an antigen.

Binding affinity between two molecules, e.g. an IL11RA binding region of an IL11RA binding polypeptide or IL11RA inhibitor, such as a hIL11RA binding region of a hIL11RA binding polypeptide or hIL11RA inhibitor as disclosed herein, and IL11RA, through a monovalent interaction may be quantified by determining the equilibrium dissociation constant (K_{D}). K_{D} can be determined by measurement of the kinetics of complex formation and dissociation, e.g. by the Surface Plasmon Resonance (SPR) method.

The rate constants corresponding to the association and the dissociation of a monovalent complex are referred to as the association rate constant k_{d} (or kₒₙ) and dissociation rate constant k_{d} (or k_{off}), respectively. K_{D} is related to k_{d} and k_{d} through the equation K_{D}=k_{d}/kₐ. Following the above definition, binding affinities associated with different molecular interactions, such as comparison of the binding affinity of different binders for a given antigen, may be compared by comparison of the K_{D} values for the individual binder/antigen complexes (such as the interaction between the compounds as disclosed herein and hIL11RA). The K_{D} of an IL11RA binding region of an IL11RA binding polypeptide or IL11RA inhibitor, such as a hIL11RA binding region of a hIL11RA binding polypeptide or hIL11RA inhibitor for its target may be less than 100 μM such as less than 10 μM, such as less than 9 UM, such as less than 8 UM, such as less than 7 UM, such as less than 6 μM, such as less than 5 μM, such as less than 4 μM, such as less than 3 μM, such as less than 2 μM, such as less than 1 μM, such as less than 0.9 μM, such as less than 0.8 UM, such as less than 0.7 μM, such as less than 0.6 UM, such as less than 0.5 μM, such as less than 0.4 μM, such as less than 0.3 μM, such as less than 0.2 μM, such as less than 0.1 μM, such as less than 20 nM, such as less than 10 nM, such as less than 2 nM, such as less than 1 nM.

Preferably the K_{D} is less than 0.1 μM, such as less than 10 nM, such as less than 2 nM, such as less than 1 nM, such as less than 0.5 nM. More preferably the K_{D} is less than 0.1 nM.

The term “binding motif” (BM) as used herein refers to an IL11RA binding region of an IL11RA binding polypeptide or IL11RA inhibitor, such as a hIL11RA binding region of a hIL11RA binding polypeptide or hIL11RA inhibitor. The (BM) comprises key binding residues enabling tight binding to IL11RA and forms part of e.g. a three-helix bundle protein domain. For example, the (BM) may constitute two alpha helices with an interconnecting loop, within said three-helix bundle protein domain.

In one embodiment the (BM) comprises 25-28 amino acid residues. In a preferred embodiment the (BM) comprises 27 amino acid residues.

In one embodiment the (BM) sequence is represented by SEQ ID NO:3 comprising fixed and non-fixed (variable) amino acid residues.

In one embodiment the (BM) sequence corresponds to the sequence from position 9 to position 35 (both positions included) in a sequence selected from the group consisting of SEQ ID NOs: 7-11; or a sequence having at least 90% identity thereto, such as at least 91%, at least 92%, at least 93% or greater identity thereto, such as at least 94%, at least 95%, at least 96%, at least 97%, at least 98% or at least 99% identity to said (BM).

In some embodiments the (BM) is characterized in comprising amino acid residues which are either fixed or not fixed (variable). The non-fixed amino acid residues are identified herein by Xₓ, wherein the letter in subscript identifies the position in the (BM).

N- and C-terminally the (BM) is flanked by non-(BM) amino acid residues and taken together the (BM) and non-(BM) residues make up a hIL11RA binding polypeptide or hIL11RA inhibitor.

The term “biparatopic” as used herein is used to characterize a binder, e.g. an IL11RA inhibitor, that has two distinct paratopes and can recognize and bind to two different epitopes on a given target molecule. The paratopes can simultaneously interact with two different epitopes on one target molecule (or two separate target molecules), enhancing binding specificity and functional activity through avidity.

A “cross-species reactive” polypeptide binds to IL11RA from more than one species (e.g. human, dog, pig and cynomolgus monkey) with comparable affinity, in particular with a Ko in the range of a factor of 100, such as within a range of a factor of 50, within a range of a factor of 20, or within a range of a factor of 10. Within a K_{D} range of a defined factor X means that the highest affinity for a particular listed species is not more than X-times higher than the lowest affinity measured for binding to a different listed species. A person skilled in the art will understand that any method for measuring affinity can be used to verify that a cross-species reactive polypeptide binds to the target antigen from all listed species within a given K_{D} factor range as described herein as long as the same conditions are applied to the K_{D} measurement for all listed species. Preferably, the K_{D} values are measured using SPR, in particular at 25° C.

The term “epitope” as used herein is defined in the context of a molecular interaction between an “antigen binding polypeptide”, such as a hIL11RA binding polypeptide, such as the hIL11RA binding polypeptide and hIL11RA inhibitors as disclosed herein, and its corresponding antigen. Generally, “epitope” refers to the area or region on an antigen to which a binder binds, i.e. the area or region in physical contact with the binder. Physical contact may be defined using various criteria (e.g. a distance cut-off of 4-5 Å, such as 4, 4.5 or 5 Å; or solvent accessibility) for atoms in the binding molecule and antigen molecules. hIL11RA may comprise a number of different epitopes, which may include, without limitation, (1) linear peptide epitopes (2) conformational epitopes which consist of one or more non-contiguous amino acids located near each other in the hIL11RA conformation.

The epitope for a given binder/antigen pair can be described and characterized at different levels of detail using a variety of experimental and computational epitope mapping methods. The experimental methods include mutagenesis, X-ray crystallography, Nuclear Magnetic Resonance (NMR) spectroscopy, Hydrogen Deuterium eXchange Mass Spectrometry (HDX-MS), Fast photochemical oxidation of proteins (FPOP), various competition binding methods and computational methods such as Alphafold 2; all methods that are known in the art.

In the context of an X-ray derived crystal structure defined by spatial coordinates of a complex between a binding molecule, e.g. an IL11RA binding polypeptide as disclosed herein, and IL11RA, the term epitope is herein, unless otherwise specified or contradicted by context, specifically defined as hIL11RA residues characterized by having a heavy atom (i.e. a non-hydrogen atom) within a distance of 3.5 Å, from a heavy atom in the binding molecule. Epitopes described at the amino acid level, e.g. determined from an X-ray structure, are said to be identical if they contain the same set of amino acid residues. Epitopes are said to overlap if at least one amino acid residue is shared by the epitopes. Epitopes are said to be separate (unique) if no amino acid residue is shared by the epitopes.

The epitope may be identified by routine methods. For example, the general location of an epitope may be determined by assessing the ability of hIL11RA binding polypeptides to bind to different fragments or variants of hIL11RA. The specific amino acids within hIL11RA that make contact with a binder (epitope) may also be determined using routine methods. For example, the binder and target molecule may be combined and the resulting complex may be crystallised. The crystal structure of the complex may be determined and used to identify specific sites of interaction between the antibody and its target. Epitope residues which are common to several different compounds may be regarded as ‘key’ epitope residues.

The terms “fusion” and “fused” as used herein refers to a compound comprising two or more individually defined polypeptides which are covalently linked by a peptide bond.

The term “half-life” or “half-life in blood” as used herein refers to the time required for half the quantity of a substance administered to a patient to be metabolized or eliminated from the blood of the patient by normal biological processes. Typically, half-life is assessed based on blood plasma samples.

The term “half-life extending moiety” as used herein refers to a moiety capable of extending the half-life in blood of the compound to which it is attached. A half-life extending moiety comprises a “half-life extender” and an optional “linker”. The half-life extender or half-life extending moiety thus serves the purpose of the extending half-life in blood of the compound as disclosed herein and in particular the IL11RA inhibitors as disclosed herein.

A half-life extending moiety may consist of one half-life extender (and no linker).

A half-life extending moiety may comprise one linker and one half-life extender.

A half-life extending moiety may comprise one linker and two or more half-life extenders.

When the linker is present, the half-life extending moiety attaches to the hIL11RA inhibitor backbone via the linker. When the linker is absent, the half-life extender attaches directly to the polypeptide backbone.

In preferred embodiment the half-life extending moiety is preferably attached to the hIL11RA inhibitor by genetic fusion (i.e., the half-life extended hIL11RA polypeptide is generated by translation of a nucleic acid in which a polynucleotide encoding the hIL11RA inhibitor is joined in-frame with a polynucleotide encoding the half-life extending moiety, such as an ABD, or ABD and linker) to one another. In such embodiments the half-life extending moiety is said to be ‘fused’ to the hIL11RA inhibitor. In one embodiment the half-life extending moiety is attached via a linker. In one embodiment the half-life extending moiety is attached without a linker.

In another embodiment the half-life extending moiety can be attached by way of chemical conjugation after the hIL11RA inhibitor has been expressed. In such embodiments the half-life extending moiety is preferably covalently attached to the side chain of a surface exposed lysine or a cysteine residue in the polypeptide after the polypeptide as such has been produced (e.g. by synthesis or recombinant expression), preferably via the epsilon-amino group.

In some embodiments the two different methods of attaching a half-life extending moiety can be combined.

In some embodiments the half-life extending moiety is capable of non-covalently binding to albumin, thereby promoting the circulation of the hIL11RA inhibitor in the blood stream and prolonging its half-life. Thus, in one embodiment the half-life extender is an albumin-binding domain (ABD). In one embodiment the ABD comprises or consists of the sequence GEGDLAEAKEAANAELDSYGVSDFYKKLIDKAKTVEGVEALKDAILAALP (SEQ ID NO:13).

In other embodiments the half-life extender may comprise an acyl group. The half-life extender may comprise a fatty acyl group. The acyl group may be branched or unbranched. The acyl group may be saturated or unsaturated.

The half-life extender may comprise a distal carboxylic acid group.

The half-life extender may comprise at least two acidic groups, wherein one acidic group is attached terminally.

The half-life extender may comprise a fatty acid group.

The half-life extender may comprise a fatty acid group and an amide group.

The half-life extender may comprise a distal carboxylic acid group and an amide group.

The half-life extender may comprise an alkyl group.

The half-life extender may comprise an aryl group.

The half-life extender may comprise a tetrazole group.

The half-life extender may comprise a sulfonic acid group.

The half-life extender may comprise a phenoxy group.

The half-life extender may comprise a benzoic acid group.

The half-life extender may comprise 8-30 carbon atoms. The half-life extender may comprise 8, 9, 10, 11, 12, 13, 14, 15, 16, 17, 18, 19, 20, 21, 22, 23, 24, 25, 26, 27, 28, 29 or 30 carbon atoms.

The half-life extender may comprise 6-30 consecutive —CH₂— groups. The half-life extender may comprise a carbon chain comprising at least 6, 7, 8, 9, 10, 11, 12, 13, 14, 15, 16, 17, 18, 19, 20, 21, 22, 23, 24, 25, 26, 27, 28, 29 or 30 consecutive —CH₂— groups.

The half-life extender may comprise 12-26 carbon atoms. The half-life extension moiety may comprise 12, 13, 14, 15, 16, 17, 18, 19, 20, 21, 22, 23, 24, 25 or 26 carbon atoms.

The half-life extender may comprise 10-26 consecutive —CH₂— groups. The half-life extender may comprise a carbon chain comprising 10, 11, 12, 13, 14, 15, 16, 17, 18, 19, 20, 21, 22, 23, 24, 25 or 26 consecutive —CH₂— groups.

The half-life extender may comprise 16-22 carbon atoms. The half-life extension moiety may comprise 16, 17, 18, 19, 20, 21 or 22 carbon atoms.

The half-life extender may comprise 14-20 consecutive —CH₂— groups. The half-life extender may comprise a carbon chain comprising 14, 15, 16, 17, 18, 19 or 20 consecutive —CH₂— groups.

The half-life extender may comprise 16-22 consecutive carbon atoms and 14-20 consecutive-CH₂-groups.

The half-life extender may comprise 16 consecutive carbon atoms and 14 consecutive —CH₂— groups.

The half-life extender may comprise 18 consecutive carbon atoms and 16 consecutive —CH₂— groups.

The half-life extender may comprise 20 consecutive carbon atoms and 18 consecutive —CH₂— groups.

The half-life extender may comprise 22 consecutive carbon atoms and 20 consecutive —CH₂— groups.

The half-life extender may comprise a group defined by: HOOC—(CH₂)ₙ—CO—* wherein n is an integer in the range of 8-20.

The term “IC₅₀” as used herein, represents the concentration of a compound, such as an IL11RA inhibitor, that reduces a given response in an assay half-way between the maximal response and the baseline.

The term “IL11RA-based signalling” as used herein refers to IL11-induced hexameric complex formation between IL11, IL11RA and gp130 through which intracellular downstream signalling is elicited/initiated.

The term “IL11RA binding polypeptide” as used herein refers to a polypeptide which is capable of binding to IL11RA. Such polypeptide may or may not inhibit the biological activity of IL11RA, such as hIL11RA as represented by SEQ ID NO:1.

The term “hIL11RA binding polypeptide” as used herein refers to a polypeptide which is capable of binding to hIL11RA. Such polypeptide may or may not inhibit the biological activity of hIL11RA as represented by SEQ ID NO:1.

The term “IL11RA inhibitor” as used herein refers to a polypeptide which is capable of binding to one or more epitopes on IL11RA and capable of inhibiting the biological activity, such as IL11RA-based signalling, of IL11RA such as, for example, hIL11RA as represented by SEQ ID NO:1.

The term “hIL11RA inhibitor” as used herein refers to a polypeptide which is capable of binding to one or more epitopes hIL11RA and capable of inhibiting the biological activity, such as hIL11RA-based signalling, of hIL11RA as represented by SEQ ID NO:1.

In one embodiment the IL11RA inhibitor, such as a hIL11RA inhibitor, binds to two different epitopes.

In one embodiment the IL11RA inhibitor, such as a hIL11RA inhibitor, binds to two different non-overlapping epitopes.

In one embodiment the IL11RA inhibitor, such as a hIL11RA inhibitor, does not comprise a half-life extending moiety.

In a preferred embodiment the IL11RA inhibitor, such as a hIL11RA inhibitor, comprises one or more half-life extending moiety (ies).

The terms “inhibition” or “inhibit” refer to a decrease or cessation of any phenotypic characteristic (such as binding, a biological activity or function) or to the decrease or cessation in the incidence, degree, or likelihood of that characteristic. The “inhibition” needs not to be complete as long as it is detectable using an appropriate assay. In some embodiments, by “reduce” or “inhibit” is meant the ability to cause a decrease of 20% or greater. In another embodiment, by “reduce” or “inhibit” is meant the ability to cause a decrease of 50% or greater. In yet another embodiment, by “reduce” or “inhibit” is meant the ability to cause an overall decrease of 75%, 85%, 90%, 95%, or greater.

The term “identity” as known in the art, refers to a relationship between the sequences of two or more polypeptides, as determined by comparing the sequences. In the art, “identity” also means the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. In the present invention identity was determined using Needleman (Needleman et al. J. Mol. Biol. 1970; 48:443-453) from EMBOSS-6.6.0 using the parameters 10 and 0.5 for gaps opening and extensions, respectively (gapopen=10, gapextend=0.5). The scores for each sequence in a pair are calculated as the number of matches in the aligned sequence, normalized by the sequence length and multiplied by 100.

The term “linker” as used herein refers to at least one atom that forms a covalent bond between chemical entities. If the chemical entities are linked solely through peptide bonds, the linker can also be referred to as a “peptide linker”. Otherwise, the linker can also be referred to as a “chemical linker”.

An example of an IL11RA inhibitor is two IL11RA binding polypeptides connected via a linker. Another example of an IL11RA inhibitor is two IL11RA binding polypeptides and one half-life-extending moiety individually and serially connected N- or C-terminally via linkers.

In one embodiment the linkers are identical and another embodiment the linkers are different. IL11RA inhibitors—in some instances comprising a half-life-extending moiety—may for example have the general modular structure (N- to C-terminal):
“First IL11RA binding polypeptide”- “linker”-“second IL11RA binding polypeptide”, or
“Second IL11RA binding polypeptide”- “linker”−“first IL11RA binding polypeptide, or
“Half-life extending moiety”−“linker”−“first IL11RA binding polypeptide”−“linker”−“second IL11RA binding polypeptide”, or
“Half-life extending moiety”−“linker”−“second IL11RA binding polypeptide”−“linker”−“first IL11RA binding polypeptide”, or
“First IL11RA binding polypeptide”−“linker”−“second IL11RA binding polypeptide”−“linker”-“half-life extending moiety”, or
“Second IL11RA binding polypeptide”−“linker”−“first IL11RA binding polypeptide”−“linker”-“half-life extending moiety”.

In some embodiments a first linker and a second linker are used. For example:
“Half-life extending moiety”−“first linker”−“first IL11RA binding polypeptide”−“second linker”-“second IL11RA binding polypeptide”, or
“Half-life extending moiety”−“first linker”−“second IL11RA binding polypeptide”−“second linker”−“first IL11RA binding polypeptide”, or
“First IL11RA binding polypeptide”−“first linker”−“second IL11RA binding polypeptide”-“second linker”−“Half-life extending moiety”, or
“Second IL11RA binding polypeptide”−“first linker”−“first IL11RA binding polypeptide”-“second linker”−“half-life extending moiety”.

In one embodiment the first linker and second linker are identical.

In one embodiment the first linker and second linker are not identical.

Preferably identical linkers are used.

The linker may for example be composed of an amino acid sequence, comprising no or multiple repeats.

For example, a linker may comprise 2 to 30 amino acid residues, 3 to 30 amino acid residues, 4 to 24 amino acid residues, or 4 to 16 amino acid residues.

Non-limiting examples of linkers include *-GAQP-* (SEQ ID NO:14), *-GGGS-* (SEQ ID NO: 15), *-GEQP-* (SEQ ID NO: 16), *-GI-* linker, *-GV-* linker, *-GT-* linker, *-GL-* linker, or another amino acid composite linker. Two examples of linkers are x4 and x6 repeat composites of GAQP, being 16 and 24 amino acid residues in length, respectively. Such repeats may also be described as (GAQP)ₓ, wherein x is an integer representing the number of repeats, for example 4 or 6. Preferably the linker is (GAQP) 4 or GAQPGAQPGAQPGAQP (Chem.9/SEQ ID NO:13).

In some embodiments the linker serves as attachment point for one or more half-life extending moieties.

In preferred embodiments an IL11RA inhibitor has the following structure (N- to C-terminal): “Half-life extending moiety”−“first linker”−“first IL11RA binding polypeptide”−“second linker”-“second IL11RA binding polypeptide”, wherein the first and second linker are identical.

Preferably the first and second linker is GAQPGAQPGAQPGAQP according to SEQ ID NO: 13.

“Substitution” variants preferably involve the replacement of one or more amino acid(s) with the same number of amino acid(s). Substitutions may be, but are not limited to, conservative substitutions. For example, an amino acid may be substituted to an amino acid with similar biochemical properties, for example, a basic amino acid may be substituted to another basic amino acid (e.g. lysine to arginine), an acidic amino acid may be substituted to another acidic amino acid (e.g glutamate to aspartate), a neutral amino acid may be substituted to another neutral amino acid (e.g threonine to serine), a charged amino acid may be substituted to another charged amino acid (e.g. glutamate to aspartate), a hydrophilic amino acid may be substituted to another hydrophilic amino acid (e.g. asparagine to glutamine), a hydrophobic amino acid may be substituted to another hydrophobic amino acid (e.g. alanine to valine), a polar amino acid may be substituted to another polar amino acid (e.g. serine to threonine), an aromatic amino acid may be substituted to another aromatic amino acid (e.g. phenylalanine to tryptophan) and an aliphatic amino acid may be substituted to another aliphatic amino acid (e.g. leucine to isoleucine).

Preferred variants include those in which instead of the amino acid which appears in the sequence comprises a structural analogue of the amino acid.

### Compounds of the Present Invention

The IL11RA binding polypeptides and IL11RA inhibitors as disclosed herein offer several advantages over conventional antibodies due to their significantly smaller size—at least apx. 7-fold less than the molecular weight of an IgG antibody-allowing for enhanced penetrability deep into tissues in a manner similar to a small molecule, easier formulation, and better scalable production thereby making it possible to reach more patients. Importantly, the components (such as binding polypeptides and linkers) of the hIL11RA inhibitors can be combined in a modular fashion and certain functionalities can be added in a fit-for-purpose fashion e.g. half-life extension, via a half-life extending moiety component. The components can thus be regarded as intermediates suitable for use in the production of an IL11RA inhibitor comprising such components. A component in the form of an IL11RA binding polypeptide may—in some embodiments—in and of itself serve as an IL11RA inhibitor.

A non-limiting illustrative example of an IL11RA inhibitor of the invention is shown in FIG. 1.

In one embodiment the IL11RA inhibitors as disclosed herein comprise a component which is a three-helix bundle protein domain derived from the Z domain of staphylococcal protein A.

In one embodiment the IL11RA inhibitors as disclosed herein comprise a component which is derived from a three-helix bundle protein domain derived from Ddi1 (S. cerevisiae).

In a preferred embodiment the IL11RA inhibitors as disclosed herein comprises an IL11RA binding polypeptide which is a three-helix bundle protein domain derived from the Z domain of staphylococcal protein A and an IL11RA binding polypeptide which is a three-helix bundle protein derived from the ubiquitin-binding protein fragment from Ddi1 (S. cerevisiae) and stabilized through engineering by introduction of a cysteine-based disulphide bond.

Thus, in one embodiment the IL11RA binding polypeptides, such as those listed in Example 4 herein, can be regarded as intermediates for use in the manufacture of the IL11RA inhibitors as disclosed herein.

In one embodiment the IL11RA inhibitors as disclosed herein comprise a first and a second IL11RA binding polypeptide, wherein both IL11RA binding polypeptides are capable of binding to—and inhibiting IL11RA signalling.

In a preferred embodiment the IL11RA inhibitors as disclosed herein comprise a first and a second IL11RA binding polypeptide, wherein the first IL11RA binding polypeptide is capable of binding to—and inhibiting IL11RA signalling and wherein the second IL11RA binding polypeptide is not capable of inhibiting IL11RA signalling, but rather serves to strengthen binding of the IL11RA inhibitor molecule to IL11RA.

In one aspect the IL11RA inhibitors as disclosed herein further comprise a half-life extending moiety, preferably being an albumin-binding domain (ABD).

In one embodiment the half-life extending moiety is fused to an IL11RA inhibitor.

In preferred embodiments the albumin-binding domain is fused to an IL11RA inhibitor comprising two binders capable of binding to IL11RA.

In one embodiment the IL11RA inhibitors as disclosed herein are capable of binding to IL11RA and capable of inhibiting IL11-mediated inhibition of lipoprotein lipase (LPL).

In one aspect the present invention relates to an IL11RA binding polypeptide capable of binding an epitope comprising the amino acid residues Y125, K150, L154, A156, F187, W188, S189, Q213, Q249, H251, F252, L253, D297, F298, L299 and D300 on hIL11RA according to SEQ ID NO:1 as determined at 3.5 Å.

In one aspect the present invention relates to an IL11RA binding polypeptide comprising the amino acid residue sequence selected from

i)
A₁S₂A₃T₄F₅P₆X₇Q₈C₉X₁₀X₁₁Q₁₂L₁₃M₁₄D₁₅L₁₆G₁₇F₁₈P₁₉X₂₀Y₂₁A₂₂V₂₃X₂₄A₂₅A₂6L₂₇X₂₈
Y₂₉T₃₀N₃₁G₃₂N₃₃C₃₄E₃₅X₃₆A₃₇A₃₈S₃₉L₄₀L₄₁F₄₂X₄₃R₄₄ according to SEQ ID NO: 2

wherein, independently of each other,
X₇ is R, V or I;
X₁₀ is A, L, D, E, T, V, I or Y;
X₁₁ is W, A, R or F;
X₂₀ is Q, A, R, V, L or F;
X₂₄ is I, A, H, V, L, F or Y;
X₂₈ is R, H, V, L, I, F or Y;
X₃₆ is R, A, H, T, Q, V, L, I, F or Y;
X₄₃ is A or H;
and
ii) an amino acid residue sequence having at least 90%, at least 91%, at least 92%, at least 93%, at least 94%, at least 95%, at least 96%, at least 97%, at least 98% or at least 99% identity to the sequence defined in i), wherein said polypeptide is an IL11RA inhibitor.

In one aspect the present invention relates to an IL11RA binding polypeptide capable of binding an epitope comprising the amino acid residues P141, R143, L145, P167, W168, P169, L175, V198, N199, P200 and A203 on hIL11RA according to SEQ ID NO:1 as determined at 3.5 Å.

As described in detail in the experimental section herein, the analysis of IL11RA binding polypeptides and IL11RA inhibitors comprising such binding polypeptides—as disclosed herein—has led to the identification of a number of individual binding motif (BM) sequences.

Thus, in one aspect the present invention relates to an IL11RA binding polypeptide comprising a binding motif (BM), said (BM) consisting of an amino acid residue sequence selected from

iii)
XₐWX_{b}AWX_{c}EIX_{d}XₑLPNLNX₁WQX_{g}AAFIXₕSLXᵢ according to SEQ ID NO: 3

wherein, independently of each other,
Xₐ is A or L;
X_{b} is Q or A;
X_{c} is D or A;
X_{d} is D, A, T, V, I or L;
Xₑ is H, A, R, S, T, Q, I, L, F or Y;
X_{f} is P or A;
X_{g} is K, A, T or I;
Xₕ is L or A;
Xᵢ is L or A;
and
iv) an amino acid residue sequence which has at least 90%, at least 91%, at least 92%, at least 93%, at least 94%, at least 95%, at least 96%, at least 97%, at least 98% or at least 99% identity to the sequence defined in iii).

“Variable” (non-fixed) positions as used herein, are positions denoted with an “X” in sequences such as i) and iii) as defined above, and in v) further below.

The sequence of individual binding motifs corresponds to amino acid positions 9-35 in e.g. SEQ ID NO:4 and 7-11 as presented in the sequence listing.

The function of the IL11RA inhibitors as disclosed herein, is primarily dependent on the structure of the region of the polypeptide that facilitates binding to its target, i.e. the Binding Motif (BM) where a region is defined as such.

In certain binding polypeptides, such as—but not limited to—those derived from a Ddi1 three-helix scaffold, the (BM) spans nearly the entirety of the binding polypeptide and is therefore embodied in the sequence of the binding polypeptide as a whole (and not singled out).

Thus, IL11RA inhibitors may for example comprise two IL11RA binding polypeptides, for which the (BM) is only specifically defined for one of the binding polypeptides.

In some embodiments, the (BM) as defined above “forms part of” a three-helix bundle protein domain. This is understood to mean that the sequence of the (BM) is “inserted” into or “grafted” onto the sequence of the original three-helix bundle domain, such that the (BM) replaces a similar structural motif in the original domain. For example, without wishing to be bound by theory, the (BM) is thought to constitute two of the three helices of a three-helix bundle and can therefore replace such a two-helix motif within any three-helix bundle. As the skilled person will realize, the replacement of two helices of the three-helix bundle domain by the two (BM) helices has to be performed so as not to affect the basic structure of the polypeptide. That is, the overall folding of the Ca backbone of the polypeptide according to this embodiment of the invention is substantially the same as that of the three-helix bundle protein domain of which it forms a part, e.g. having the same elements of secondary structure in the same order etc. Thus, a (BM) according to the present disclosure “forms part” of a three-helix bundle domain if the polypeptide according to this embodiment has the same fold as the original domain, implying that the basic structural properties are shared, those properties e.g. resulting in similar CD spectra. The skilled person is aware of other parameters that are relevant.

In particular embodiments, the binding motif (BM) of the IL11RA binding polypeptide or IL11RA inhibitor, thus forms part of a three-helix bundle protein domain. For example, the (BM) may essentially constitute two alpha helices with an interconnecting loop, within said three-helix bundle protein domain. In particular embodiments, said three-helix bundle protein domain is selected from domains of bacterial receptor proteins. Non-limiting examples of such domains are the five different three-helical domains of Protein A from Staphylococcus aureus, such as domain B, and derivatives thereof. In some embodiments, the three-helical bundle protein domain is a variant of protein Z, which is derived from domain B of staphylococcal Protein A (Wahlberg E et al. 2003, PNAS 100 (6): 3185-3190).

It is possible to make minor changes to the sequence of amino acid residues outside the (BM) in a polypeptide without affecting the function thereof. Thus, the disclosure encompasses modified variants of the inhibitors, which have retained IL11RA, such as hIL11RA, binding characteristics.

Thus, in one aspect the present invention relates to an IL11RA binding polypeptide comprising the amino acid residue sequence selected from

v)
V₁D₂N₃R₄F₅N₆X₇E₈-(BM)-X₃₆D₃₇P₃₈S₃₉Q₄₀W₄₁A₄₂N₄₃L₄₄X
L₄₅X₄₆E₄₇A₄₈R₄₉R₅₀L₅₁N₅₂D₅₃A₅₄Q₅₅A₅₆P₅₇X₅₈
according to SEQ ID NO: 4

wherein, independently of each other,
X₇ is R or E;
X₃₆ is D or E;
X₄₆ is A or E;
X₅₈ is R or E;
and
vi) an amino acid residue sequence having at least 90%, at least 91%, at least 92%, at least 93%, at least 94%, at least 95%, at least 96%, at least 97%, at least 98% or at least 99% identity to the sequence defined in v).

In this way, encompassed by the present disclosure is an IL11RA inhibitor comprising an amino acid sequence with 90% or greater identity, such as 91%, 92%, 93%, 94%, 95%, 96%, 97%, 98% or 99% identity, to an inhibitor as defined in the Examples herein such as an inhibitor having SEQ ID NO:19 (Chem. 12) or SEQ ID NO:20 (Chem. 13).

Also encompassed by the present disclosure is an IL11RA inhibitor comprising an amino acid sequence with 90% or greater identity, such as at least 91%, at least 92%, at least 93%, at least 94%, at least 95%, at least 96%, at least 97%, at least 98% or at least 99% identity, to an inhibitor as defined in the Examples herein such as an inhibitor having SEQ ID NO:19 (Chem. 12) or SEQ ID NO:20 (Chem.13).

For example, it is possible that an amino acid residue belonging to a certain functional grouping of amino acid residues (e.g. hydrophobic, hydrophilic, polar etc) could be exchanged for another amino acid residue from the same functional group.

In one aspect the present invention relates to an IL11RA inhibitor comprising a first hIL11RA binding polypeptide capable of binding an epitope comprising the amino acid residues Y125, K150, L154, A156, F187, W188, S189, Q213, Q249, H251, F252, L253, D297, F298, L299 and D300 on hIL11RA according to SEQ ID NO:1 as determined at 3.5 Å, and a second hIL11RA binding polypeptide capable of binding an epitope comprising the amino acid residues P141, R143, L145, P167, W168, P169, L175, V198, N199, P200 and A203 on hIL11RA according to SEQ ID NO:1 as determined at 3.5 Å.

In one embodiment the IL11RA inhibitor binds to two different non-overlapping epitopes on hIL11RA.

In one aspect the present invention relates to an IL11RA inhibitor comprising a first IL11RA binding polypeptide comprising the amino acid residue sequence selected from

i)
A₁S₂A₃T₄F₅P₆X₇Q₈C₉X₁₀X₁₁Q₁₂L₁₃M₁₄D₁₅L₁₆G₁₇F₁₈P₁₉
X₂₀Y₂₁A₂₂V₂₃X₂₄A₂₅A₂₆L₂₇X₂₈Y₂₉T₃₀N₃₁G₃₂N₃₃C₃₄E₃₅
X₃₆A₃₇A₃₈S₃₉L₄₀L₄₁F₄₂X₄₃R₄₄ according to
SEQ ID NO: 2

wherein, independently of each other,
X₇ is R, V or I;
X₁₀ is A, L, D, E, T, V, I or Y;
X₁₁ is W, A, R or F;
X₂₀ is Q, A, R, V, L or F;
X₂₄ is I, A, H, V, L, For Y;
X₂₈ is R, H, V, L, I, F or Y;
X₃₆ is R, A, H, T, Q, V, L, I, F or Y;
X₄₃ is A or H;
and
ii) an amino acid residue sequence having at least 90%, at least 91%, at least 92%, at least 93%, at least 94%, at least 95%, at least 96%, at least 97%, at least 98% or at least 99% identity to the sequence defined in i);
and comprising
a second IL11RA binding polypeptide comprising a binding motif (BM), said (BM) consisting of an amino acid residue sequence selected from

iii)
XₐWX_{b}AWX_{c}EIX_{d}XₑLPNLNX_{f}WQX_{g}AAFIXₕSLXᵢ
according to SEQ ID NO: 3

wherein, independently of each other,
Xₐ is A or L;
X_{b} is Q or A;
X_{c} is D or A;
X_{d} is D, A, T, V, I or L;
Xₑ is H, A, R, S, T, Q, I, L, F or Y;
X_{f} is P or A;
X_{g} is K, A, T or I;
Xₕ is L or A;
Xᵢ is L or A;
and
iv) an amino acid residue sequence which has at least 90%, at least 91%, at least 92%, at least 93%, at least 94%, at least 95%, at least 96%, at least 97%, at least 98% or at least 99% identity to the sequence defined in iii).

In a preferred embodiment the second IL11RA binding polypeptide comprises the amino acid residue sequence selected from

v)
V₁D₂N₃R₄F₅N₆X₇E₈-(BM)-X₃₆D₃₇P₃₈S₃₉Q₄₀W₄₁A₄₂N₄₃L₄₄
L₄₅X₄₆E₄₇A₄₈R₄₉R₅₀L₅₁N₅₂D₅₃A₅₄Q₅₅A₅₆P₅₇X₅₈
according to SEQ ID NO: 4

wherein, independently of each other,
X₇ is R or E;
X₃₆ is D or E;
X₄₆ is A or E;
X₅₈ is R or E;
and
an amino acid residue sequence having at least 90%, at least 91%, at least 92%, at least 93%, at least 94%, at least 95%, at least 96%, at least 97%, at least 98% or at least 99% identity to the sequence defined in v).

In a preferred embodiment the IL11RA inhibitor comprises a first IL11RA binding polypeptide selected from the group consisting of:

a)
ASATFPRQCAWQLMDLGFPQYAVIAALRYTNGNCERAASLLFAR
according to SEQ ID NO: 5;
and
b)
ASATFPRQCAWQLMDLGFPQYAVIAALRYTNGNCERAASLLFHR
according to SEQ ID NO: 6;

and
a second IL11RA binding polypeptide selected from the group consisting of:

c)
VDNRFNREAWQAWDEIDHLPNLNPWQKAAFILSLLDDPSQWANLLAEARR
LNDAQAPR according to SEQ ID NO: 7;
d)
VDNRFNREAWQAWDEIDHLPNLNPWQKAAFILSLLDDPSQWANLLEEARR
LNDAQAPR according to SEQ ID NO: 8;
e)
VDNRFNREAWQAWDEIDHLPNLNPWQKAAFILSLLEDPSQWANLLEEARR
LNDAQAPE according to SEQ ID NO: 9;
f)
VDNRFNEEAWQAWDEIDHLPNLNPWQKAAFILSLLDDPSQWANLLEEARR
LNDAQAPE according to SEQ ID NO: 10;
and
g)
VDNRFNEEAWQAWDEIDHLPNLNPWQKAAFILSLLEDPSQWANLLEEARR
LNDAQAPE according to SEQ ID NO: 11.

In preferred embodiments the IL11RA binding polypeptide or IL11RA inhibitor comprises a half-life extending moiety, for example, but not limited to the amino acid residue sequence GEGDLAEAKEAANAELDSYGVSDFYKKLIDKAKTVEGVEALKDAILAALP according to SEQ ID NO: 12, preferably fused to the binding polypeptide or inhibitor.

In some aspect of the present invention relates to an IL11RA inhibitor comprising an amino acid residue sequence selected from the group consisting of:

h)
GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFARGAQPGAQPGAQPGAQPVDNRFNREAWQAWDEIDHLPNLNP
WQKAAFILSLLDDPSQWANLLAEARRLNDAQAPR according to
SEQ ID NO: 17 (Chem. 10);
i)
GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFHRGAQPGAQPGAQPGAQPVDNRFNREAWQAWDEIDHLPNLNP
WQKAAFILSLLDDPSQWANLLEEARRLNDAQAPR according to
SEQ ID NO: 18 (Chem. 11);
j)
GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFARGAQPGAQPGAQPGAQPVDNRFNREAWQAWDEIDHLPNLNP
WQKAAFILSLLEDPSQWANLLEEARRLNDAQAPE according to
SEQ ID NO: 19 (Chem. 12);
k)
GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFARGAQPGAQPGAQPGAQPVDNRFNEEAWQAWDEIDHLPNLNP
WQKAAFILSLLDDPSQWANLLEEARRLNDAQAPE according to
SEQ ID NO: 20 (Chem. 13);
and
l)
GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFARGAQPGAQPGAQPGAQPVDNRFNEEAWQAWDEIDHLPNLNP
WQKAAFILSLLEDPSQWANLLEEARRLNDAQAPE according to
SEQ ID NO: 21 (Chem. 14).

In one embodiment the IL11RA inhibitor comprises
the amino acid residue sequence
GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFARGAQPGAQPGAQPGAQPVDNRFNREAWQAWDEIDHLPNLNP
WQKAAFILSLLDDPSQWANLLAEARRLNDAQAPR according to
SEQ ID NO: 17 (Chem. 10).
In one embodiment the IL11RA inhibitor comprises
the amino acid residue sequence
GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFHRGAQPGAQPGAQPGAQPVDNRFNREAWQAWDEIDHLPNLNP
WQKAAFILSLLDDPSQWANLLEEARRLNDAQAPR according to
SEQ ID NO: 18 (Chem. 11).
In one embodiment the IL11RA inhibitor comprises
the amino acid residue sequence
GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFARGAQPGAQPGAQPGAQPVDNRFNREAWQAWDEIDHLPNLNP
WQKAAFILSLLEDPSQWANLLEEARRLNDAQAPE according to
SEQ ID NO: 19 (Chem. 12).
In one embodiment the IL11RA inhibitor comprises
the amino acid residue sequence
GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFARGAQPGAQPGAQPGAQPVDNRFNEEAWQAWDEIDHLPNLNP
WQKAAFILSLLDDPSQWANLLEEARRLNDAQAPE according to
SEQ ID NO: 20 (Chem. 13).
In one embodiment the IL11RA inhibitor comprises
the amino acid residue sequence
GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFARGAQPGAQPGAQPGAQPVDNRFNEEAWQAWDEIDHLPNLNP
WQKAAFILSLLEDPSQWANLLEEARRLNDAQAPE according to
SEQ ID NO: 21 (Chem. 14).

In a preferred embodiment the IL11RA inhibitor is a hILRA11 inhibitor.

In one embodiment a hILRA11 inhibitor of the invention is capable of inhibiting hIL11-mediated intracellular signalling as determined according to the method of Example 2 herein.

In one embodiment a hILRA11 inhibitor of the invention is capable of inhibiting hIL11-mediated intracellular signalling with a potency (IC₅₀) of 1.0 nM or less as determined according to the method of Example 2 herein.

In one embodiment a hILRA11 inhibitor of the invention is capable of inhibiting hIL11-mediated intracellular signalling with a potency (IC₅₀) of 0.5 nM or less as determined according to the method of Example 2 herein.

In one aspect the preset invention provides a kit comprising (i) the inhibitor or a pharmaceutical composition comprising said inhibitor, and (ii) instructions for use.

### Production Process

The compounds as disclosed herein may be produced by means of recombinant nucleic acid techniques. In general, a cloned wild-type compounds as disclosed herein nucleic acid sequence is modified to encode the desired protein. This modified sequence is then inserted into an expression vector, which is in turn transformed or transfected into host cells.

The nucleic acid construct encoding the compounds as disclosed herein may suitably be of genomic, cDNA or synthetic origin. Amino acid sequence alterations are accomplished by modification of the genetic code by well-known techniques.

The DNA sequences encoding the compounds as disclosed herein are usually inserted into a recombinant vector which may be any vector, which may conveniently be subjected to recombinant DNA procedures, and the choice of vector will often depend on the host cell into which it is to be introduced. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the compounds as disclosed herein is operably linked to additional segments required for transcription of the DNA. The term, “operably linked” indicates that the segments are arranged so that they function in concert for their intended purposes, e.g. transcription initiates in a promoter and proceeds through the DNA sequence coding for the polypeptide until it terminates within a terminator.

Thus, expression vectors for use in expressing compounds as disclosed herein will comprise a promoter capable of initiating and directing the transcription of a cloned gene or cDNA. The promoter may be any DNA sequence, which shows transcriptional activity in the host cell of choice and may be derived from genes encoding proteins either homologous or heterologous to the host cell.

Additionally, expression vectors for use of expression of compounds as disclosed herein will also comprise a terminator sequence, a sequence recognized by a host cell to terminate transcription. The terminator sequence is operably linked to the 3′ terminus of the nucleic acid sequence encoding the polypeptide. Any terminator which is functional in the host cell of choice may be used in the present invention.

Expression of compounds as disclosed herein can be aimed for either intracellular expression in the cytosol of the host cell or be directed into the secretory pathway for extracellular expression into the growth medium.

Intracellular expression is the default pathway and requires an expression vector with a DNA sequence comprising a promoter followed by the DNA sequence encoding the compounds as disclosed herein polypeptide followed by a terminator.

To direct the compounds as disclosed herein into the secretory pathway of the host cells, a secretory signal sequence (also known as signal peptide or a pre sequence) is needed as an N-terminal extension of the compounds as disclosed herein. A DNA sequence encoding the signal peptide is joined to the 5′ end of the DNA sequence encoding the compounds as disclosed herein in the correct reading frame. The signal peptide may be that normally associated with the protein or may be from a gene encoding another secreted protein.

The procedures used to ligate the DNA sequences coding for the compounds as disclosed herein, the promoter, the terminator and/or secretory signal sequence, respectively, and to insert them into suitable vectors containing the information necessary for replication, are well known to persons skilled in the art (cf., for instance, Sambrook et al., Molecular Cloning: A Laboratory Manual, Cold Spring Harbor, New York, 1989).

In one embodiment of the invention a four amino acid residue motif “GEGD” was introduced N-terminally and was observed to result in increased expression of the IL11RA inhibitors as specifically disclosed herein.

The host cell into which the DNA sequences encoding compounds as disclosed herein is introduced may be any cell that is capable of expressing compounds as disclosed herein either intracellularly or extracellularly. If posttranslational modifications are needed, suitable host cells include yeast, fungi, insects and higher eukaryotic cells such as mammalian cells.

Alternatively, the compounds as disclosed herein may be produced by means of solid-phase peptide synthesis.

### Pharmaceutical Compositions and Administration Thereof.

The compounds of the invention such as the hIL11RA inhibitors as disclosed herein may be prepared in pharmaceutical compositions. In some embodiments such composition comprises at least one pharmaceutically acceptable excipient.

The excipients may serve various purposes, e.g. as a carrier, vehicle, filler, binder, lubricant, glidant, disintegrant, flow control agent, crystallization inhibitors, solubilizer, stabilizer, colouring agent, flavouring agent, surfactant, emulsifier, delivery agent, hydrotrope or combinations thereof and/or to improve administration, and/or absorption of the active pharmaceutical ingredient(s).

The amount of each excipient used may vary within ranges conventional in the art. Techniques and excipients which may be used are described in e.g Remington: The Science and Practice of Pharmacy, 23^{nd} edition, Remington, Elsevier Science Publishing Co Inc (2020).

The compositions containing the compounds as disclosed herein can be administered for prophylactic and/or in some embodiments on-demand treatments.

The compositions are typically administered to a subject already suffering from a disease, such as the indications described below, in an amount sufficient to cure, alleviate or partially arrest the disease and its complications. An amount adequate to accomplish this is defined as “therapeutically effective amount”. As will be understood by the person skilled in the art amounts effective for this purpose will depend on the severity of the disease as well as the weight and general state of the subject.

A compound as disclosed herein, such as a hIL11RA inhibitor, may be administered parenterally, such as intravenously, such as intramuscularly, such as subcutaneously in an appropriate pharmaceutical composition.

In some embodiments the dose of the compounds to be delivered by e.g. subcutaneous or intravenous administration may be from 0.01 mg to 700 mg of the compound per day depending on the severity of the condition.

A suitable dose may also be adjusted for a particular compound based on the properties of that compound, including its in vivo half-life or mean residence time and its biological activity.

For example, compounds to be delivered could in one embodiment be administered once daily. In one embodiment the present invention relates to an injection device with a content of said composition.

In one embodiment the present invention relates to a kit comprising (i) a compound as disclosed herein, such as an IL11RA inhibitor, and (ii) instructions for use.

### Indications

The disease or disorder treatable by administering compounds as disclosed herein such as the IL11RA inhibitors as disclosed herein is any disease or condition which is improved, ameliorated, inhibited or prevented, or its occurrence rate reduced, compared to that without treatment with IL11RA inhibitors (e.g., hIL11-mediated diseases or disorders), by removing, inhibiting, reducing, or otherwise interfering with, hIL11RA activity.

Examples of diseases or disorders treatable include, but are not limited to, those involving metabolic-dysfunction associated steatohepatitis (MASH), cardiovascular disease (CVD), fibrosis, inflammatory and autoimmune diseases and cancers.

The compounds as disclosed herein and in particular the hIL11RA inhibitors as disclosed herein are also useful in the prevention or treatment of diseases or disorders associated with or resulting from MASH, CVD (such as but limited to (atrial fibrillation, syncope, edema, tachycardia, congestive heart failure), and tissue fibrosis (in lung, kidney, and heart). Certain cases of cancers are associated with increased expression of IL11 and/or IL11RA which can lead to increased cellular growth, migration, and invasion. Such cancers can arise in any tissue where IL11 and/or IL11RA are expressed (such as but not limited to liver, kidney, heart, lung, and skin). Regarding Inflammatory diseases, IL11 and/or IL11RA activation can lead to inflammation in; lung (such as asthma, chronic obstructive pulmonary disorder COPD), skin (dermatitis), ulcerative colitis, nephritis, infection-induced inflammation, and CVD such as atherosclerosis and diabetes.

In some embodiments, the fibrosis can be found in the lung (idiopathic pulmonary fibrosis), liver (MASH), kidney (CKD, DKD), heart (myocardial fibrosis), skin, and intestine.

### FURTHER EMBODIMENTS

1. An IL11RA binding polypeptide capable of binding an epitope comprising one or more of the amino acid residues Y125, K150, L154, A156, F187, W188, S189, Q213, Q249, H251, F252, L253, D297, F298, L299 and D300 on hIL11RA according to SEQ ID NO:1 as determined at 3.5 Å.
2. An IL11RA binding polypeptide capable of binding an epitope comprising the amino acid residues Y125, K150, L154, A156, F187, W188, S189, Q213, Q249, H251, F252, L253, D297, F298, L299 and D300 on hIL11RA according to SEQ ID NO:1 as determined at 3.5 Å.
3. An IL11RA binding polypeptide comprising the amino acid residue sequence selected from

i)
A₁S₂A₃T₄F₅P₆X₇Q₈C₉X₁₀X₁₁Q₁₂L₁₃M₁₄D₁₅L₁₆G₁₇F₁₈P₁₉
X₂₀Y₂₁A₂₂V₂₃X₂₄A₂₅A₂₆L₂₇X₂₈Y₂₉T₃₀N₃₁G₃₂N₃₃C₃₄E₃₅
X₃₆A₃₇A₃₈S₃₉L₄₀L₄₁F₄₂X₄₃R₄₄ according to
SEQ ID NO: 2

wherein, independently of each other,
X₇ is R, V or I;
X₁₀ is A, L, D, E, T, V, I or Y;
X₁₁ is W, A, R or F;
X₂₀ is Q, A, R, V, L or F;
X₂₄ is I, A, H, V, L, F or Y;
X₂₈ is R, H, V, L, I, F or Y;
X₃₆ is R, A, H, T, Q, V, L, I, For Y;
X₄₃ is A or H;
and
ii) an amino acid residue sequence having at least 90%, at least 91%, at least 92%, at least 93%, at least 94%, at least 95%, at least 96%, at least 97%, at least 98% or at least 99% identity to the sequence defined in i).
4. The IL11RA binding polypeptide according to any one of the preceding embodiments, wherein said polypeptide comprises an amino acid residue sequence selected from

iii)
ASATFPRQCAWQLMDLGFPQYAVIAALRYTNGNCERAASLLFAR
according to SEQ ID NO: 5;
and
ASATFPRQCAWQLMDLGFPQYAVIAALRYTNGNCERAASLLFHR
according to SEQ ID NO: 6;

and
iv) an amino acid residue sequence having at least 90%, at least 91%, at least 92%, at least 93%, at least 94%, at least 95%, at least 96%, at least 97%, at least 98% or at least 99% identity to the sequence defined in iii).
5. The IL11RA binding polypeptide according to any one of the preceding embodiments wherein the IL11RA binding polypeptide is a hIL11RA binding polypeptide capable of binding to hIL11RA according to SEQ ID NO:1.
6. The IL11RA binding polypeptide according to any one of the preceding embodiments wherein the IL11RA polypeptide comprises 44 to 60 residues, such as 44 to 55 residues, such as 44 to 50 residues, such as 44 to 48 residues, such as 44 to 46 residues.
7. The IL11RA binding polypeptide according to any one of the preceding embodiments wherein the polypeptide comprises 44, 45, 46, 47, 48, 49 or 50 residues.
8. The IL11RA binding polypeptide according to any one of the preceding embodiments wherein the IL11RA binding polypeptide comprises 44 residues.
9. The IL11RA binding polypeptide according to any one of the preceding embodiments, wherein the IL11RA binding polypeptide comprises an amino acid residue sequence selected from

ASATFPRQCAWQLMDLGFPQYAVIAALRYTNGNCERAASLLFAR
according to SEQ ID NO: 5;
and
ASATFPRQCAWQLMDLGFPQYAVIAALRYTNGNCERAASLLFHR
according to SEQ ID NO: 6;

optionally comprising 1, 2 or 3 substitution(s).
10. The IL11RA binding polypeptide according to embodiment 9 wherein said substitution(s) is/are a conservative substitution(s).
11. The IL11RA binding polypeptide according to any one of the preceding embodiments wherein the IL11RA binding polypeptide is capable of binding IL11RA with a K_{D} value of less than 100 μM, such as less than 10 UM, such as less than 9 UM, such as less than 8 μM, such as less than 7 UM, such as less than 6 μM, such as less than 5 μM, such as less than 4 μM, such as less than 3 μM, such as less than 2 μM, such as less than 1 μM, such as less than 0.9 UM, such as less than 0.8 UM, such as less than 0.7 UM, such as less than 0.6 μM, such as less than 0.5 μM, such as less than 0.4 μM, such as less than 0.3 μM, such as less than 0.2 M, such as less than 0.1 μM, such as less than 20 nM, such as less than 10 nM, such as less than 2 nM, such as less than 1 nM.
12. The IL11RA binding polypeptide according to any one of the preceding embodiments wherein the IL11RA binding polypeptide is a cross-species reactive polypeptide.
13. The IL11RA binding polypeptide according to any one of the preceding embodiments wherein the IL11RA binding polypeptide is capable of binding to one or more of cynomolgus monkey IL11RA, dog IL11RA, human IL11RA, rat IL11RA, and pig IL11RA.
14. The IL11RA binding polypeptide according to any one of the preceding embodiments wherein the IL11RA binding polypeptide is a hIL11RA inhibitor.
15. The IL11RA binding polypeptide according to any one of the preceding embodiments for use as an intermediate in the manufacture of an IL11RA inhibitor comprising a first and a second hIL11RA binding polypeptide, such as a biparatopic IL11RA inhibitor.
16. An IL11RA binding polypeptide capable of binding an epitope comprising one or more of the amino acid residues P141, R143, L145, P167, W168, P169, L175, V198, N199, P200 and A203 on hIL11RA according to SEQ ID NO:1 as determined at 3.5 Å.
17. An IL11RA binding polypeptide capable of binding an epitope comprising the amino acid residues P141, R143, L145, P167, W168, P169, L175, V198, N199, P200 and A203 on hIL11RA according to SEQ ID NO:1 as determined at 3.5 Å.
18. The IL11RA binding polypeptide according to embodiment 16 or 17 comprising a binding motif (BM), said (BM) consisting of an amino acid residue sequence selected from

v)
XₐWX_{b}AWX_{c}EIX_{d}XₑLPNLNX_{f}WQX_{g}AAFIXₕSLXᵢ according
to SEQ ID NO: 3

wherein, independently of each other,
Xₐ is A or L;
X_{b} is Q or A;
X_{c} is D or A;
X_{d} is D, A, T, V, I or L;
Xₑ is H, A, R, S, T, Q, I, L, F or Y;
X_{f} is P or A;
X_{g} is K, A, T or I;
Xₕ is L or A;
Xᵢ is L or A;
and
vi) an amino acid residue sequence which has at least 90%, at least 91%, at least 92%, at least 93%, at least 94%, at least 95%, at least 96%, at least 97%, at least 98% or at least 99% identity to the sequence defined in v).
19. An IL11RA binding polypeptide comprising a binding motif (BM), said (BM) consisting of an amino acid residue sequence selected from

vii)
XₐWX_{b}AWX_{c}EIX_{d}XₑLPNLNX_{f}WQX_{g}AAFIXₕSLXᵢ according
to SEQ ID NO: 3

wherein, independently of each other,
Xₐ is A or L;
X_{b} is Q or A;
X_{c} is D or A;
X_{d} is D, A, T, V, I or L;
Xₑ is H, A, R, S, T, Q, I, L, F or Y;
X_{f} is P or A;
X_{g} is K, A, T or I;
Xₙ is L or A;
Xᵢ is L or A;
and
viii) an amino acid residue sequence which has at least 90%, at least 91%, at least 92%, at least 93%, at least 94%, at least 95%, at least 96%, at least 97%, at least 98% or at least 99% identity to the sequence defined in vii).
20. The IL11RA binding polypeptide according to embodiment 18 or 19 wherein the (BM) is AWQAWDEIDHLPNLNPWQKAAFILSLL (SEQ ID NO:31),
   optionally comprising 1, 2, or 3 substitution(s), wherein said substitution(s) can take place in any one of positions 1, 3, 6, 9, 10, 16, 19, 24 and/or 27 in the (BM) sequence.
21. The IL11RA binding polypeptide according to any one of embodiments 18-20 comprising the amino acid residue sequence selected from

ix)
V₁D₂N₃R₄F₅N₆X₇E₈-(BM)-X₃₆D₃₇P₃₈S₃₉Q₄₀W₄₁A₄₂N₄₃L₄₄
L₄₅X₄₆E₄₇A₄₈R₄₉R₅₀L₅₁N₅₂D₅₃A₅₄Q₅₅A₅₆P₅₇X₅₈
according to SEQ ID NO: 4

wherein, independently of each other,
X₇ is R or E;
X₃₆ is D or E;
X₄₆ is A or E;
X₅₈ is R or E;
and
x) an amino acid residue sequence having at least 90%, at least 91%, at least 92%, at least 93%, at least 94%, at least 95%, at least 96%, at least 97%, at least 98% or at least 99% identity to the sequence defined in ix).
22. The IL11RA binding polypeptide according to any one of embodiments 16-21 wherein the IL11RA binding polypeptide is a hIL11RA binding polypeptide capable of binding to hIL11RA according to SEQ ID NO:1.
23. The IL11RA binding polypeptide according to any one of embodiments 16-22 wherein the polypeptide comprises 58 to 70 amino acid residues, such as 58 to 65 amino acid residues, such as 58 to 62 amino acid residues, such as 58 to 60 amino acid residues.
24. The IL11RA binding polypeptide according to any one of embodiments 16-23 wherein the polypeptide comprises 58, 59, 60, 61, 62, 63, 64, or 65 amino acid residues.
25. The IL11RA binding polypeptide according to any one of embodiments 16-24 wherein the polypeptide comprises 58 amino acid residues.
26. The IL11RA binding polypeptide according to any one of embodiments 16-25 wherein the polypeptide is capable of binding hIL11RA with a K_{D} value of less than 100 UM, such as less than 10 UM, such as less than 9 UM, such as less than 8 M, such as less than 7 μM, such as less than 6 μM, such as less than 5 M, such as less than 4 μM, such as less than 3 μM, such as less than 2 μM, such as less than 1 μM, such as less than 0.9 μM, such as less than 0.8 M, such as less than 0.7 UM, such as less than 0.6 μM, such as less than 0.5 μM, such as less than 0.4 μM, such as less than 0.3 μM, such as less than 0.2 μM, such as less than 0.1 μM, such as less than 20 nM, such as less than 10 nM, such as less than 1 nM, such as less than 0.1 nM.
27. The hIL11RA binding polypeptide according to any one of embodiments 16-26 wherein said inhibitor comprises the amino acid residue sequence

VDNRFNREAWQAWDEIDHLPNLNPWQKAAFILSLLDDPSQWANLLAEARR
LNDAQAPR according to SEQ ID NO: 7;
or
VDNRFNREAWQAWDEIDHLPNLNPWQKAAFILSLLDDPSQWANLLEEARR
LNDAQAPR according to SEQ ID NO: 8;
or
VDNRFNREAWQAWDEIDHLPNLNPWQKAAFILSLLEDPSQWANLLEEARR
LNDAQAPE according to SEQ ID NO: 9;
or
VDNRFNEEAWQAWDEIDHLPNLNPWQKAAFILSLLDDPSQWANLLEEARR
LNDAQAPE according to SEQ ID NO: 10;
or
VDNRFNEEAWQAWDEIDHLPNLNPWQKAAFILSLLEDPSQWANLLEEARR
LNDAQAPE according to SEQ ID NO: 11;

optionally comprising 1, 2 or 3 substitution(s).
28. The IL11RA binding polypeptide according to embodiment 27 wherein said substitution is a conservative substitution.
29. The IL11RA binding polypeptide according to any one of embodiments 16-28 wherein the polypeptide is a cross-species reactive polypeptide.
30. The IL11RA binding polypeptide according to embodiment 29 wherein the polypeptide is capable of binding to one or more of cynomolgus monkey IL11RA, dog IL11RA, human IL11RA, rat IL11RA and pig IL11RA.
31. The IL11RA binding polypeptide according to any one of embodiments 16-30 wherein the polypeptide is a hIL11RA inhibitor.
32. The IL11RA binding polypeptide according to any one of embodiments 16-31 for use as an intermediate in the manufacture of an IL11RA inhibitor comprising a first and a second IL11RA binding polypeptide, such as a biparatopic IL11RA binding polypeptide.
33. An IL11RA inhibitor comprising a first IL11RA binding polypeptide according to any one of embodiments 1-15 and a second IL11RA binding polypeptide according to any one of embodiments 16-32 and optionally a linker linking said first and second polypeptides.
34. An IL11RA inhibitor comprising
   a first IL11RA binding polypeptide capable of binding an epitope comprising one or more of the amino acid residues Y125, K150, L154, A156, F187, W188, S189, Q213, Q249, H251, F252, L253, D297, F298, L299 and D300,
   and
   a second IL11RA binding polypeptide capable of binding an epitope comprising one or more of the amino acid residues P141, R143, L145, P167, W168, P169, L175, V198, N199, P200 and A203;
   on hIL11RA according to SEQ ID NO:1 as determined at 3.5 Å.
35. An IL11RA inhibitor comprising
   a first IL11RA binding polypeptide capable of binding an epitope comprising the amino acid residues Y125, K150, L154, A156, F187, W188, S189, Q213, Q249, H251, F252, L253, D297, F298, L299 and D300,
   and
   a second IL11RA binding polypeptide capable of binding an epitope comprising the amino acid residues P141, R143, L145, P167, W168, P169, L175, V198, N199, P200 and A203;
   on hIL11RA according to SEQ ID NO:1 as determined at 3.5 Å.
36. An IL11RA inhibitor comprising
   a first IL11RA binding polypeptide comprising the amino acid residue sequence selected from

xi)
A₁S₂A₃T₄F₅P₆X₇Q₈C₉X₁₀X₁₁Q₁₂L₁₃M₁₄D₁₅L₁₆G₁₇F₁₈P₁₉X₂₀Y₂₁A₂₂V₂₃
X₂₄A₂₅A₂₆L₂₇X₂₈Y₂₉T₃₀N₃₁G₃₂N₃₃C₃₄E₃₅X₃₆A₃₇A₃₈S₃₉L₄₀L₄₁F₄₂X₄₃R₄₄
according to SEQ ID NO: 2

wherein, independently of each other,
X₇ is R, V or I;
X₁₀ is A, L, D, E, T, V, I or Y;
X₁₁ is W, A, R or F;
X₂₀ is Q, A, R, V, L or F;
X₂₄ is I, A, H, V, L, F or Y;
X₂₈ is R, H, V, L, I, F or Y;
X₃₆ is R, A, H, T, Q, V, L, I, F or Y;
X₄₃ is A or H;
and
xii) an amino acid residue sequence having at least 90%, at least 91%, at least 92%, at least 93%, at least 94%, at least 95%, at least 96%, at least 97%, at least 98% or at least 99% identity to the sequence defined in xi);
and
a second binding polypeptide comprising a binding motif (BM) which consists of an amino acid residue sequence selected from

xiii)
XₐWX_{b}AWX_{c}EIX_{d}XₑLPNLNX_{f}WQX_{g}AAFIXₕSLXᵢ
according to SEQ ID NO: 3

wherein, independently of each other,
Xₐ is A or L;
X_{b} is Q or A;
X_{c} is D or A;
X_{d} is D, A, T, V, I or L;
Xₑ is H, A, R, S, T, Q, I, L, F or Y;
X_{f} is P or A;
X_{g} is K, A, T or I;
Xₕ is L or A;
Xᵢ is L or A;
and
xiv) an amino acid residue sequence which has at least 90%, at least 91%, at least 92%, at least 93%, at least 94%, at least 95%, at least 96%, at least 97%, at least 98% or at least 99% identity to the sequence defined in xiii).
37. The IL11RA inhibitor according to any one of embodiments 33-36, wherein the (BM) is AWQAWDEIDHLPNLNPWQKAAFILSLL (SEQ ID NO:31), optionally comprising 1, 2, or 3 substitution(s), wherein said substitution(s) can take place in any one of positions 1, 3, 6, 9, 10, 16, 19, 24, and/or 27 in the (BM).
38. The IL11RA inhibitor according to any one of embodiments 33-37, wherein the second binding polypeptide comprises the amino acid residue sequence selected from

xv)
V₁D₂N₃R₄F₅N₆X₇E₈-(BM)-X₃₆D₃₇P₃₈S₃₉Q₄₀W₄₁A₄₂N₄₃L₄₄L₄₅
X₄₆E₄₇A₄₈R₄₉R₅₀L₅₁N₅₂D₅₃A₅₄Q₅₅A₅₆P₅₇X₅₈
according to SEQ ID NO:4

wherein, independently of each other,
X₇ is R or E;
X₃₆ is D or E;
X₄₆ is A or E;
X₅₈ is R or E;
and
xvi) an amino acid residue sequence having at least 90%, at least 91%, at least 92%, at least 93%, at least 94%, at least 95%, at least 96%, at least 97%, at least 98% or at least 99% identity to the sequence defined in xv).
39. The IL11RA binding polypeptide or IL11RA inhibitor according to any one of the preceding embodiments further comprising a half-life extending moiety.
40. The IL11RA binding polypeptide or IL11RA inhibitor according to embodiment 39 wherein said half-life extending moiety is a polypeptide.
41. The IL11RA binding polypeptide or IL11RA inhibitor according to any one of the preceding embodiments wherein said polypeptide or inhibitor comprises a half-life extending moiety comprising the amino acid residue sequence GEGDLAEAKEAANAELDSYGVSDFYKKLIDKAKTVEGVEALKDAILAALP according to SEQ ID NO:12.
42. The IL11RA binding polypeptide or IL11RA inhibitor according to any one of embodiments 39-41 wherein said half-life extending moiety is fused or conjugated to said inhibitor.
43. The IL11RA inhibitor according to any one of embodiments 33-42 having the formula (N- to C-terminal)
   “First IL11RA binding polypeptide”−“second IL11RA binding polypeptide”, or
   “Second IL11RA binding polypeptide”−“first IL11RA binding polypeptide, or
   “First IL11RA binding polypeptide”−“second linker”−“second IL11RA binding polypeptide”, or
   “Second IL11RA binding polypeptide”−“second linker”−“first IL11RA binding polypeptide, or
   “Half-life extending moiety”−“first linker”−“first IL11RA binding polypeptide”−“second linker”−“second IL11RA binding polypeptide”, or
   “Half-life extending moiety”−“first linker”−“second IL11RA binding polypeptide”−“second linker”−“first IL11RA binding polypeptide”, or
   “First IL11RA binding polypeptide”−“first linker”−“second IL11RA binding polypeptide”-“second linker”−“Half-life extending moiety”, or
   “Second IL11RA binding polypeptide”−“first linker”−“first IL11RA binding polypeptide”-“second linker”−“half-life extending moiety”.
44. The IL11RA inhibitor according to any one of embodiments 33-43 having the formula (N- to C-terminal)
   “Half-life extending moiety”−“first linker”−“first IL11RA binding polypeptide”−“second linker”−“second IL11RA binding polypeptide”.
45. The IL11RA inhibitor according to any one of embodiments 33-44, wherein said IL11RA inhibitor comprises
   a first IL11RA binding polypeptide selected from the group consisting of:

a) ASATFPRQCAWQLMDLGFPQYAVIAALRYTNGNCERAASLLFAR
according to SEQ ID NO: 5;
and
b) ASATFPRQCAWQLMDLGFPQYAVIAALRYTNGNCERAASLLFHR
according to SEQ ID NO: 6;

and
a second IL11RA binding polypeptide selected from the group consisting of:

c) VDNRFNREAWQAWDEIDHLPNLNPWQKAAFILSLLDDPSQWANLLAE
ARRLNDAQAPR according to SEQ ID NO: 7;
d) VDNRFNREAWQAWDEIDHLPNLNPWQKAAFILSLLDDPSQWANLLEE
ARRLNDAQAPR according to SEQ ID NO: 8;
e) VDNRFNREAWQAWDEIDHLPNLNPWQKAAFILSLLEDPSQWANLLEE
ARRLNDAQAPE according to SEQ ID NO: 9;
f) VDNRFNEEAWQAWDEIDHLPNLNPWQKAAFILSLLDDPSQWANLLEE
ARRLNDAQAPE according to SEQ ID NO: 10;
and
g) VDNRFNEEAWQAWDEIDHLPNLNPWQKAAFILSLLEDPSQWANLLEE
ARRLNDAQAPE according to SEQ ID NO: 11.

46. The IL11RA inhibitor according to any one of embodiments 43-45 wherein the first and/or second linker(s) is/are (GAQP)ₓ, wherein X is an integer in the range 1-10.
47. The IL11RA inhibitor according to any one of embodiments 43-46 wherein the first and/or second linker(s) is/are GAQPGAQPGAQPGAQP according to SEQ ID NO:13.
48. The IL11RA inhibitor according to any one of embodiments 43-47 wherein said IL11RA inhibitor comprises a second linker which connects the first IL11RA binding polypeptide and the second IL11RA binding polypeptide.
49. The IL11RA inhibitor according to embodiment 48 wherein the second linker connects the C-terminal amino acid residue of the first IL11RA binding polypeptide with the N-terminal amino acid residue of the second IL11RA binding polypeptide.
50. The IL11RA inhibitor according to any one of embodiments 43-48 wherein the second linker connects the C-terminal amino acid residue of the second IL11RA binding polypeptide with the N-terminal amino acid residue of the first IL11RA binding polypeptide.
51. The IL11RA inhibitor according to any one of embodiments 43-48, wherein said IL11RA inhibitor comprises a first linker which connects the C-terminal amino acid residue of the half-life extending moiety with the N-terminal amino acid residue of the first hIL11RA binding polypeptide.
52. The IL11RA inhibitor according to any one of embodiments 33-51 wherein the IL11RA inhibitor is capable of binding IL11RA with a K_{D} value of less than 100 UM, such as less than 10 μM, such as less than 9 UM, such as less than 8 UM, such as less than 7 UM, such as less than 6 μM, such as less than 5 μM, such as less than 4 μM, such as less than 3 μM, such as less than 2 μM, such as less than 1 μM, such as less than 0.9 UM, such as less than 0.8 μM, such as less than 0.7 μM, such as less than 0.6 μM, such as less than 0.5 μM, such as less than 0.4 μM, such as less than 0.3 μM, such as less than 0.2 μM, such as less than 0.1 μM, such as less than 10 nM, such as less than 1 nM, such as less than 0.5 nM, preferably less than 0.1 nM.
53. The IL11RA inhibitor according to any one of embodiments 33-52 wherein said IL11RA inhibitor comprises an amino acid residue sequence selected from the group consisting of

a) GEGDLAEAKEAANAELDSYGVSDFYKKLIDKAKTVEGVEALKDAILA
ALPGAQPGAQPGAQPGAQPASATFPRQCAWQLMDLGFPQYAVIAALRYTN
GNCERAASLLFARGAQPGAQPGAQPGAQPVDNRFNREAWQAWDEIDHLPN
LNPWQKAAFILSLLDDPSQWANLLAEARRLNDAQAPR according to
SEQ ID NO: 17 (Chem. 10);
b) GEGDLAEAKEAANAELDSYGVSDFYKKLIDKAKTVEGVEALKDAILA
ALPGAQPGAQPGAQPGAQPASATFPRQCAWQLMDLGFPQYAVIAALRYTN
GNCERAASLLFHRGAQPGAQPGAQPGAQPVDNRFNREAWQAWDEIDHLPN
LNPWQKAAFILSLLDDPSQWANLLEEARRLNDAQAPR according to
SEQ ID NO: 18 (Chem. 11);
c) GEGDLAEAKEAANAELDSYGVSDFYKKLIDKAKTVEGVEALKDAILA
ALPGAQPGAQPGAQPGAQPASATFPRQCAWQLMDLGFPQYAVIAALRYTN
GNCERAASLLFARGAQPGAQPGAQPGAQPVDNRFNREAWQAWDEIDHLPN
LNPWQKAAFILSLLEDPSQWANLLEEARRLNDAQAPE according to
SEQ ID NO: 19 (Chem. 12);
d) GEGDLAEAKEAANAELDSYGVSDFYKKLIDKAKTVEGVEALKDAILA
ALPGAQPGAQPGAQPGAQPASATFPRQCAWQLMDLGFPQYAVIAALRYTN
GNCERAASLLFARGAQPGAQPGAQPGAQPVDNRFNEEAWQAWDEIDHLPN
LNPWQKAAFILSLLDDPSQWANLLEEARRLNDAQAPE according to
SEQ ID NO: 20 (Chem. 13);
and
e) GEGDLAEAKEAANAELDSYGVSDFYKKLIDKAKTVEGVEALKDAILA
ALPGAQPGAQPGAQPGAQPASATFPRQCAWQLMDLGFPQYAVIAALRYTN
GNCERAASLLFARGAQPGAQPGAQPGAQPVDNRFNEEAWQAWDEIDHLPN
LNPWQKAAFILSLLEDPSQWANLLEEARRLNDAQAPE according to
SEQ ID NO: 21 (Chem. 14).

54. The IL11RA inhibitor according to any one of embodiments 33-53 wherein said inhibitor comprises
   a first IL11RA binding polypeptide binder capable of binding to a first epitope on hIL11RA,
   a second IL11RA binding polypeptide binder capable of binding to a second epitope on hIL11RA, and
   a half-life extending moiety
   wherein said first and second epitope on hIL11RA do not overlap.
55. An IL11RA inhibitor capable of binding to hIL11RA according to SEQ ID NO:1,
   wherein said inhibitor comprises an amino acid residue sequence
   which has at least 90%, at least 91%, at least 92%, at least 93%, at least 94%, at least 95%, at least 96%, at least 97%, at least 98% or at least 99% identity to SEQ ID NO:19.
56. A hIL11RA inhibitor capable of binding to hIL11RA according to SEQ ID NO:1 wherein said inhibitor comprises the amino acid residue sequence

GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFARGAQPGAQPGAQPGAQPVDNRFNREAWQAWDEIDHLPNLNP
WQKAAFILSLLDDPSQWANLLAEARRLNDAQAPR according to
SEQ ID NO: 17 (Chem. 10).

57. A hIL11RA inhibitor capable of binding to hIL11RA according to SEQ ID NO:1 wherein said inhibitor comprises the amino acid residue sequence

GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFHRGAQPGAQPGAQPGAQPVDNRFNREAWQAWDEIDHLPNLNP
WQKAAFILSLLDDPSQWANLLEEARRLNDAQAPR according to
SEQ ID NO: 18 (Chem. 11).

58. A hIL11RA inhibitor capable of binding to hIL11RA according to SEQ ID NO:1 wherein said inhibitor comprises the amino acid residue sequence

GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFARGAQPGAQPGAQPGAQPVDNRFNREAWQAWDEIDHLPNLNP
WQKAAFILSLLEDPSQWANLLEEARRLNDAQAPE according to
SEQ ID NO: 19 (Chem. 12).

59. A hIL11RA inhibitor capable of binding to hIL11RA according to SEQ ID NO:1 wherein said inhibitor comprises the amino acid residue sequence

GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFARGAQPGAQPGAQPGAQPVDNRFNEEAWQAWDEIDHLPNLNP
WQKAAFILSLLDDPSQWANLLEEARRLNDAQAPE according to
SEQ ID NO: 20 (Chem. 13).

60. A hIL11RA inhibitor capable of binding to hIL11RA according to SEQ ID NO:1 wherein said inhibitor comprises the amino acid residue sequence

GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFARGAQPGAQPGAQPGAQPVDNRFNEEAWQAWDEIDHLPNLNP
WQKAAFILSLLEDPSQWANLLEEARRLNDAQAPE according to
SEQ ID NO: 21 (Chem. 14).

61. A compound comprising the amino acid residue sequence

GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFARGAQPGAQPGAQPGAQPVDNRFNREAWQAWDEIDHLPNLNP
WQKAAFILSLLDDPSQWANLLAEARRLNDAQAPR according to
SEQ ID NO: 17 (Chem. 10).

62. A compound comprising the amino acid residue sequence

GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFHRGAQPGAQPGAQPGAQPVDNRFNREAWQAWDEIDHLPNLNP
WQKAAFILSLLDDPSQWANLLEEARRLNDAQAPR according to
SEQ ID NO: 18 (Chem. 11).

63. A compound comprising the amino acid residue sequence

GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFARGAQPGAQPGAQPGAQPVDNRFNREAWQAWDEIDHLPNLNP
WQKAAFILSLLEDPSQWANLLEEARRLNDAQAPE according to
SEQ ID NO: 19 (Chem. 12).

64. A compound comprising the amino acid residue sequence

GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFARGAQPGAQPGAQPGAQPVDNRFNEEAWQAWDEIDHLPNLNP
WQKAAFILSLLDDPSQWANLLEEARRLNDAQAPE according to
SEQ ID NO: 20 (Chem. 13).

65. A compound comprising the amino acid residue sequence

GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFARGAQPGAQPGAQPGAQPVDNRFNEEAWQAWDEIDHLPNLNP
WQKAAFILSLLEDPSQWANLLEEARRLNDAQAPE according to
SEQ ID NO: 21 (Chem. 14).

66. The compound according to embodiment 65 wherein said compound is an IL11RA inhibitor or a hIL11RA inhibitor.
67. A pharmaceutical composition comprising the IL11RA inhibitor or compound according to any one of embodiments 33-66 and one or more pharmaceutically acceptable excipients.
68. The composition according to embodiment 67 wherein the composition is a liquid composition, such as an aqueous composition.
69. The binding polypeptide, inhibitor, compound or composition according to any one of embodiments 1-68 for use in medicine.
70. The IL11RA binding polypeptide, IL11RA inhibitor, compound or composition according to any one of embodiments 1-68 for use in the treatment of MASH, CVD, fibro-inflammatory disease or fibrotic disease.
71. The IL11RA binding polypeptide, IL11RA inhibitor, compound or composition according to embodiment 70 for use in the treatment of atrial fibrillation, syncope, edema, tachycardia, or congestive heart failure.
72. A method of treating a subject suffering from MASH, CVD or fibrosis, comprising administering to said subject the binding polypeptide, inhibitor, compound or composition according to any one of embodiments 1-68.
73. A method of treating a subject according to embodiment 72 wherein said subject is suffering from atrial fibrillation, syncope, edema, tachycardia, or congestive heart failure.
74. Use of the IL11RA inhibitor, compound or composition according to any one of embodiments 33-68 for the manufacture of a medicament for the treatment of MASH, CVD, fibro-inflammatory disease or fibrotic disease.
75. Use of the IL11RA inhibitor, compound or composition according to embodiment 74 for the manufacture of a medicament for the treatment of atrial fibrillation, syncope, edema, tachycardia, or congestive heart failure.
76. A kit comprising (i) the IL11RA binding polypeptide, IL11RA inhibitor, compound or composition according to any one of embodiments 1-68 and (ii) instructions for use.
77. A polynucleotide encoding the IL11RA binding polypeptide, IL11RA inhibitor or compound according to any one of embodiments 1-66.
78. An expression vector comprising the polynucleotide according to embodiment 77.
79. A host cell which produces the IL11RA binding polypeptide, IL11RA inhibitor or compound according to any one of embodiments 1-66.
80. A method of producing an IL11RA binding polypeptide, IL11RA inhibitor or compound according to any one of embodiments 1-66 comprising
   a) culturing a host cell according to the preceding embodiment under conditions permissive of expression of the IL11RA binding polypeptide, IL11RA inhibitor or compound encoded by the expression vector of embodiment 78,
   b) isolating said IL11RA binding polypeptide, IL11RA inhibitor or compound,
   c) optionally attaching a half-life extending moiety to said isolated IL11RA binding polypeptide, IL11RA inhibitor or compound.
81. An IL11RA binding polypeptide, IL11RA inhibitor or compound obtainable by the method of the preceding embodiment.

### EXAMPLES

### List of Abbreviations

ABD Albumin-binding domain
ADA Anti-drug antibodies
AUC Area under the plasma concentration-time curves
CV Column volume
CIP Clean in place
HEPES 4-(2-hydroxyethyl)-1-piperazineethanesulfonic acid
HSA Human serum albumin
IMAC Immobilized metal affinity chromatography
i.v. Intravenous
LCMS Liquid chromatography mass spectrometry
LLOQ Lower limit of quantification
LOCI
Luminescent oxygen channeling immunoassay
MQ Milli-Q
MASH Metabolic dysfunction-associated steatohepatitis
NaCl Sodium chloride
NCA Non-compartmental analysis
PBS Phosphate buffered saline
RGA Reporter gene assay
r.t. Room temperature
RU Response units
S.C. Subcutaneous
RoA Route of administration
SPR Surface plasmon resonance
TFA Trifluoroacetic acid
TMDD Target mediated drug disposition
UPLC Ultra performance liquid chromatography

### General Methods of Preparation of Compounds

Expression of Compounds in S. cerevisiae

Plasmids for expression of the compounds as disclosed herein in S. cerevisiae were constructed by subcloning synthetic DNA fragments encoding the compounds as disclosed herein (obtained from Geneart AG (Regensburg, Germany)) into a yeast multicopy vector derived from cPOT-type expression plasmids previously described in EP171142. The resulting plasmids were transformed into yFI3313, a proprietary S. cerevisiae strain, using Frozen-EZ yeast transformation II Kit (Zymo Research, CA) according to manufacturer's instructions or other standard yeast transformation methods. Yeast transformants were selected by glucose utilization as carbon source on agar plates containing 1% yeast extract, 2% peptone, and 2% glucose. Yeast strains containing plasmids encoding said compounds were cultivated in minimal media as described by Verduyn et al. (Verduyn, C., Postma, E., Scheffers, W. A., Van Dijken, J. P. (1992) Yeast 8, 501-517) with the addition of 7 g/L yeast extract and 210 g/L glucose. Yeast supernatants with secreted hIL11RA inhibitor molecules were harvested by centrifugation.

### Purification and Analysis of IL11-Ri Variants

Variants of hIL11RA inhibitors tagged with (HisAla)₈ and non-tagged compounds were purified. (HisAla)₈ tagged hIL11RA inhibitor variants were purified by immobilized metal affinity chromatography (IMAC) on Ni-Excel resin in batch form, eluting with either Imidazole or acidic elution (conditions in Table 1). The resulting pool was polished by Reverse Phase chromatography under acidic conditions (see description in Table 2). When needed, the (HisAla)₈ tag was removed in the IMAC elution pool by cleavage with SplB, prior the polishing step. Conditions for SplB cleavage: Adjust to pH 7-8, Incubate with 1% (w/w) SplB at 5° C. for 1 day, or until cleavage was achieved.

TABLE 1
Purification of (HisAla)₈ tagged hIL11RA inhibitor variants-
Procedure for IMAC batch chromatography
IMAC chromatography

| Parameter | Information |
|---|---|
| Resin | NI-SEPHAROSE ™ excel (Ni-Excel), CYTIVA ™ |
| Solvents | A: 15 mM TriEthanolamine, 0.5M NaCl pH 7.5 |
| | B1: 250 mM Imidazol, 15 mM TriEthanolamine pH 7.5 |
| | B2: 0.5M acetic acid |
| Program | Incubation with resin: Equilibration: |
| | Overnight, at 5° C., under stirring |
| | Wash |
| | Wash 1: Buffer A, 2 resin volumes. Repeated twice |
| | Wash 2: MQ water, 2 resin volumes |
| | Elution: |
| | Buffer B1 or B2: 1 resin volume, repeated three times |
| | 100% Solvent B for 2 CV |

TABLE 2
Purification of (HisAla)₈ tagged hIL11RA inhibitor variants-Procedure
for Reverse Phase chromatography under acidic conditions
Reverse phase chromatography-Acidic conditions

| Parameter | Information |
|---|---|
| Resin column | XBridge Protein BEH C4 300 Å, WATERS ™ |
| Resin bed hight | 15 cm |
| Run Temperature | r.t. |
| Solvents | A: 0.1% (W/W) TFA in MQ water |
| | B: 0.1% (W/W) TFA in Acetonitrile |
| Flow | 25 CV/h |
| Program | Equilibration: |
| | 90% Solvent A, 10% Solvent B for 4 CV |
| | Load |
| | Elution: |
| | 40% to 65% Solvent B in 13 CV. This gradient is |
| | adapted for different IL11-Ri variants |
| | 100% Solvent B for 2 CV |

Non-tagged IL11RA inhibitor variants were purified by cation exchange chromatography (see description in Table 3), followed by a polishing step by Reverse Phase chromatography on a C4 columns at neutral pH (see Table 4).

TABLE 3
Purification of non-tagged IL11RA inhibitor variants-Procedure for
Cation exchange chromatography
Cation exchange chromatography

| Parameter | Information |
|---|---|
| Resin column | SP SEPHAROSE ™ Big Beads (SP BB), CYTIVA ™ |
| Resin bed hight | >15 cm |
| Run Temperature | r.t. |
| Load | |
| Load treatment | Adjustment to pH 3.0 and conductivity 3 mS/cm |
| | with phosphoric acid |
| Buffers | A: 100 mmol/kg citric acid pH 3.0 |
| | B: 200 mmol/kg Tris pH 8.5 |
| | C: 0.5M NaOH |
| Flow | 5-15 CV/h |
| Program | Equilibration: Buffer A, 5 CV |
| | Load |
| | Wash: Buffer A, 5 CV |
| | Elution 1: |
| | 0-100% Buffer B |
| | 100% Buffer B for ≥3 CV |
| | CIP: Buffer C for 5 CV |

TABLE 4
Purification of non-tagged IL11-RA inhibitor variants-Process for
Reverse Phase Chromatography under neutral conditions
Reverse phase chromatography-Neutral conditions

| Parameter | Information |
|---|---|
| Resin column | C4, 200 Å/15 μm, FUJI ™ |
| Resin bedheight | 25 cm |
| Run | r.t. |
| Temperature | |
| Buffers | A: 100 mmol/kg NaAc, 20 mmol/kg Triethanolamin, |
| | adjusted to pH 6.5, 10% w/w EtOH |
| | B: 100 mmol/kg NaAc, 20 mmol/kg Triethanolamin, |
| | adjusted to pH 6.5, 60% w/w EtOH |
| | C: 100 mmol/kg citric acid, 70%_{w/w} EtOH |
| Flow | 10-20 CV/h |
| Program | Equilibration: |
| | Buffer A, 3 CV |
| | Load |
| | Elution: |
| | 40% to 80% Buffer B in 40 CV. This gradient is |
| | adapted for the different IL11RA inhibitor variants |
| | 100% Buffer B for 3 CV |
| | CIP: Buffer C for 5 CV |

Protein integrity, molecular masses and purity were analysed by LC-MS. LC-MS was performed using a reversed-phase ultra-high-performance liquid chromatography (for example on a BEH C4 column, 1×50 mm, Waters Corp, Milford, MA, USA) with a linear gradient of acetonitrile (for example, 10-50% of acetonitrile in 20 min) in 0.1% trifluoro acetic acid (TFA) delivered by an UPLC chromatography system (Waters Corp, Milford, MA, USA). The eluate from the UPLC column was directed to a mass spectrometer (for example an ORBITRAP FUSION LUMOS™ TRIBRID™; Thermo Fischer Scientific, Bremen, Germany) and the identity of the compounds were determined by comparing the measured monoisotopic mass with the mass calculated based on the amino acid sequence for each compound.

### Example 1: Design of Compounds and Binding Motifs (BM)

The IL11RA binding polypeptides as disclosed herein are designed molecules developed on two different three-helix bundle protein domains.

Using in vitro mRNA display the present inventors identified different monovalent polypeptides which were shown to specifically bind to and interact with hIL11RA. It was shown that some of these polypeptides blocked hIL11-induced signalling on cells expressing the hIL11RA.

A first binder was derived from the three-helix bundle protein domain from the Z domain of staphylococcal protein A, that bind to the Fc part of immunoglobulins. By randomizing selected amino acids in the range residues 9 to residue 35 on the two first helices, which constitute the Fc-binding surface, large libraries were constructed, from which hIL11RA binding polypeptides were isolated by a variety of methods. The randomization of the Fc binding surface residues in the range residues 9 to residue 35 and subsequent selection of variants lead to replacement of the Fc binding capacity with a capacity for binding hIL11RA (selection #1).

A second binder was derived from the three-helix bundle protein domain from Ddi1 (S. cerevisiae, 2MR9) which was stabilized by engineering of a disulfide bond between residues 9 and 34. By randomizing selected amino acids in the range of residue 8 to residues 37, large libraries were constructed, from which hIL11RA binding polypeptides were isolated.

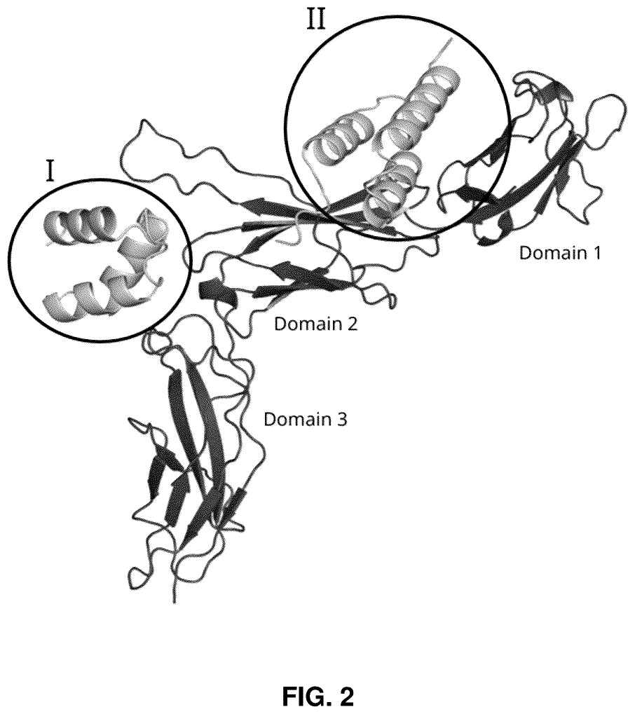

Initial hits from the selections were screened for binding to immobilized hIL11RA using Surface Plasmon Resonance (SPR). Several of the binders which were identified (e.g. SEQ ID NOs: 28-30) were then produced in larger amounts and binding was characterized using SPR and in a potency reporter gene assay (RGA) (see example 2). In addition, promising candidates were subjected to in silico epitope assessment using Alphafold 2. The in silico assessments resulted in binders being clustered to two distinct epitopes (see FIG. 2 and example 8). Selected binders were then used in competition SPR experiments to determine if they can or cannot bind to hIL11RA at the same time, or in the presence of bound hIL11.

Results from these SPR experiments suggested that two different families of binders engage hIL11RA at two distinct, non-overlapping epitopes and hence can bind to the receptor at the same time (see example 9).

In initial designs exploiting the effect of avidity two polypeptides capable of binding to hIL11RA were connected by way of several repeats of a flexible amino acid linker composed of glycines and serines (G4S-linker). While different arrangements of binding polypeptides and linkers were observed to work well, placement of a polypeptide predicted to bind to epitope “I” at the N-terminus of the construct followed by a polypeptide predicted to bind to epitope “II” (see FIG. 2 and Example 8 herein) offered superior inhibition of IL11-mediated signalling than the other way around.

In particular—in an N- to C-terminal direction—the arrangement “First hIL11RA binding polypeptide”−“Second hIL11RA binding polypeptide”, see Examples 4 and 5 herein for specific sequences, provided the highest affinity and potency.

Different linker sequences (GAQP and GEQP) and different linker length (5 repeats or 4 repeats) were subsequently tested and it was surprisingly found that a GEQP linker lead to a loss in affinity and potency compared to a GAQP linker.

In a next step the compounds were optimized for expression in S. cerevisiae by removal of sequence liabilities, e.g. known protease cleavage sites, protection of the C-terminus from carboxypeptidases.

Thus, after identification of suitable binders from the separate scaffolds, binders that target two distinct, non-overlapping epitopes were combined into one molecule by using an amino acid residue linker leading to the hIL11RA inhibitors as disclosed herein.

To optimize the pharmacokinetic properties (in vivo half-life) a polypeptide capable of binding to albumin, from herein on an “albumin-binding domain” (ABD), was attached to the compounds. N-terminal as well as C-terminal placement of the ABD was tested. N-terminal placement of the ABD proved to be more favourable for production yields and biophysical properties. Taken together the use of an ABD as a recombinant half-life extending moiety together with the biparatopic hIL11-targeting component comprising two IL11RA binding polypeptides provided a hIL11RA inhibitor with a long in vivo half-life and high potency in terms of inhibiting hIL11-mediated signalling through the hIL11RA.

The hIL11RA inhibitors can be functionalized via genetic fusion to protein modules of interest or by covalent amino acid residue side chain attachment to functional modules. Such modules include—but is not limited to—toxins, imaging agents, and/or—as described above—preferably one or more half-life extending moieties, such as an ABD.

### Example 2: Characterization of IL11RA Inhibitors with IL-11RA/STAT3-Luciferase Reporter Gene Assay

A reporter gene assay was established to determine the ability of six different IL11RA binding polypeptides to inhibit IL11 signalling (Chem. 11-16). We used luciferase under control of STAT3 response elements to measure IL11 signalling, since stimulated IL11 receptor (a hexameric complex containing a dimer of IL11, IL11RA and gp130) is known to phosphorylate STAT3, which homodimerizes and then translocates to the nucleus where it binds STAT3 response elements.

HEK293 cells (ATCC CRL-1573) were stably transfected with the STAT3-luciferase reporter plasmid (six STAT3 response elements and a TATA box inserted upstream to the luciferase gene in pGL4.20 [luc2/Puro] from PROMEGA®) and a single cell clone was isolated. This single cell clone was stably co-transfected with a human IL11RA expression plasmid based on pcDNA5/TO and a TetR expression plasmid based on pcDNA3.1/Zeo (+) and a single cell clone called clone 327 that was insensitive to tetracycline was isolated. HEK293 cells have endogenous expression of the IL11RA co-receptor, gp130.

For measuring inhibition of IL11 signaling, the HEK293/hIL11RA/TetR/STAT3-luciferase (clone 327) cells were harvested with Versene (GIBCO™) and seeded in white, opaque 384-well plates at a density of 5000 cells/well. Subsequently, compounds were added, incubated for 30 min at 37° C. and 5% CO₂ and finally 0.5 nM hIL11 (SEQ ID NO:34) was added and incubated overnight at 37° C. and 5% CO₂. Cells were lysed and luciferase substrate added using the STEADY-GLO® kit (PROMEGA®). Luminescence was detected using the ENVISION 2104™ plate reader with ultrasensitive luminescence detection (PERKIN ELMER®). Dulbecco's Modified Eagle Medium (DMEM) supplemented with 10% fetal bovine serum and 1% penicillin/streptomycin was used for seeding cells and dilution of compounds and agonist.

For determining IC₅₀ values, IL11RA inhibitor concentration-response curves were fitted to the four-parameter logistic equation and the results are presented in table 5 below.

TABLE 5
IC₅₀ values

| Chem. No | |
|---|---|
| (SEQ ID NO) | IC₅₀ (nM) |
| 11 (18) | 0.99 |
| 12 (19) | 0.31 |
| 13 (20) | 0.28 |
| 14 (21) | 0.28 |
| 15 (22) | 143.9 |
| 16 (23) | No inhibition |

Analysis of the data shows that the tested compounds having Chem. Nos 11-15 are capable of inhibiting hIL11-mediated intracellular signalling. It was further demonstrated that compounds (having high % sequence identity) inhibit hIL11-mediated intracellular signalling with a potency (IC₅₀) of 1.0 nM or less. The (Z domain-based) hIL11RA binding polypeptide designated Chem. 16 (SEQ ID NO:23) binds IL11RA but does not inhibit IL11-mediated signalling.

### Example 3: Screening of Linker Composition and Length

To determine optimal linker composition and length a panel of constructs was designed with different linker composition (repeated modules of either GAQP (SEQ ID NO:14) or GEQP (SEQ ID NO:16)) and length (four or five repeats of the four amino acid linker sequences) to link two monovalent hIL11RA binding polypeptides Chem. 15 and 16 into biparatopic molecules.

The constructs were tested in the IL11RA/STAT3-luciferase reporter gene assay as described in Example 2. For determining IC₅₀ values, biparatopic IL11RA inhibitor concentration-response curves were fitted to the four-parameter logistic equation. See table 6 below.

TABLE 6
IC₅₀ values

| Chem. No | | |
|---|---|---|
| (SEQ ID NO) | Linker | IC₅₀ (nM) |
| 17 (24) | 5xGEQP | 0.61 |
| 18 (25) | 5xGAQP | 0.23 |
| 19 (26) | 4xGEQP | 0.62 |
| 20 (27) | 4xGAQP | 0.12 |

Analysis of the data indicates that constructs with a GEQP-based linker consistently exhibit lower potency in inhibiting hIL11-mediated signalling as evidenced by higher IC₅₀-values compared to those with a GAQP-based linker. Furthermore, a 16 amino acid linker composed of four GAQP repeats (4×GAQP) (SEQ ID NO:13) appears to have higher potency compared to a 20 amino acid linker composed of five GAQP repeats (5×GAQP).

### Example 4: Monovalent hIL11RA Binding Polypeptides, Albumin-Binding Domain and Linker Sequences

TABLE 7
Non-limiting overview of sequences

| | | | |
|---|---|---|---|
| | | Used in | |
| | SEQ | IL11RA | |
| Chem. | ID | inhibitor | |
| No | NO | Chem. No | Sequence |

First hIL11RA binding polypeptides (Ddi1-based)

| | | | |
|---|---|---|---|
| 1 | 5 | 10, 12, 13, | ASATFPRQCAWQLMDLGFPQYAVIAALRYTNGNCERAASL |
| | | 14 | LFAR |
| 2 | 6 | 11 | ASATFPRQCAWQLMDLGFPQYAVIAALRYTNGNCERAASL |
| | | | LFHR |
| 15 | 22 | | ASATFPRQCAWQLMDLGFPQYAVIAALRYTNGNCERAASL |
| | | | LFKRG |
| 21 | 28 | | MASATFPRQCAWQLMDLGFPQYAVIAALRYTNGNCERAA |
| | | | SLLFKRGGGGGSGGGGSDYKDDDDKHHHHHH |

Second hIL11RA binding polypeptides (Binding Motif highlighted in underline)
(Z domain-based)

| | | | |
|---|---|---|---|
| 3, 16 | 7, 23 | 10 | VDNRFNREAWQAWDEIDHLPNLNPWQKAAFILSLLDDPSQ |
| | | | WANLLAEARRLNDAQAPR |
| 4 | 8 | 11 | VDNRFNREAWQAWDEIDHLPNLNPWQKAAFILSLLDDPSQ |
| | | | WANLLEEARRLNDAQAPR |
| 5 | 9 | 12 | VDNRFNREAWQAWDEIDHLPNLNPWQKAAFILSLLEDPSQ |
| | | | WANLLEEARRLNDAQAPE |
| 6 | 10 | 13 | VDNRFNEEAWQAWDEIDHLPNLNPWQKAAFILSLLDDPSQ |
| | | | WANLLEEARRLNDAQAPE |
| 7 | 11 | 14 | VDNRFNEEAWQAWDEIDHLPNLNPWQKAAFILSLLEDPSQ |
| | | | WANLLEEARRLNDAQAPE |
| 23 | 30 | | MVDNRFNREAWQAWDEIDHLPNLNPWQKAAFILSLLDDPS |
| | | | QWANLLAEARRLNDAQAPRGGGGSGGGGSDYKDDDDKH |
| | | | HHHHH |

Albumin-Binding Domain (ABD)

| | | | |
|---|---|---|---|
| 8 | 12 | 10-14 | GEGDLAEAKEAANAELDSYGVSDFYKKLIDKAKTVEGVEAL |
| | | | KDAILAALP |

Linker

| | | | |
|---|---|---|---|
| 9 | 13 | 10-14 | GAQPGAQPGAQPGAQP |

### Example 5: Examples of hIL11RA Inhibitor Sequences

The present example provides non-limiting examples of hIL11RA inhibitors by reference to their full sequence. ABDs are highlighted in italic linkers are shown in regular font and hIL11RA binding polypeptides are highlighted in bold.

Chem.10:
(SEQ ID NO: 17)
GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFARGAQPGAQPGAQPGAQPVDNRFNREAWQAWDEIDHLPNLNP
WQKAAFILSLLDDPSQWANLLAEARRLNDAQAPR
Chem. 11:
(SEQ ID NO: 18)
GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFHRGAQPGAQPGAQPGAQPVDNRFNREAWQAWDEIDHLPNLNP
WQKAAFILSLLDDPSQWANLLEEARRLNDAQAPR
Chem. 12:
(SEQ ID NO: 19)
GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFARGAQPGAQPGAQPGAQPVDNRFNREAWQAWDEIDHLPNLNP
WQKAAFILSLLEDPSQWANLLEEARRLNDAQAPE
Chem. 13:
(SEQ ID NO: 20)
GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFARGAQPGAQPGAQPGAQPVDNRFNEEAWQAWDEIDHLPNLNP
WQKAAFILSLLDDPSQWANLLEEARRLNDAQAPE
Chem. 14:
(SEQ ID NO: 21)
GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFARGAQPGAQPGAQPGAQPVDNRFNEEAWQAWDEIDHLPNLNP
WQKAAFILSLLEDPSQWANLLEEARRLNDAQAPE

### Example 6: Surface Plasmon Resonance (SPR) Characterization of the Interaction Between hIL11RA Inhibitors and hIL11RA

The purpose of this assay is to demonstrate that IL11RA inhibitors as disclosed herein bind to hIL11RA and to determine the binding affinity. Binding studies were executed on a BIACORE™ 8K, or BIACORE™ 8K+ (all from CYTIVA™), instruments designed to quantify molecular interactions in real-time utilizing Surface Plasmon Resonance (SPR). The experiments were conducted at 25° C., while samples were maintained at 25° C. in the sample compartment. The SPR instruments' reported signals, expressed in response units (RU), directly correlate with the mass on the sensor chip surfaces within eight parallel channels, each containing two serial flow cells.

The procedure begun with the non-covalent immobilization of Biotin CAPture reagent (CYTIVA™) onto both flow cells of a Series S Sensor Chip CAP (CYTIVA™) following the manufacturer's guidelines. Subsequently, biotinylated, recombinant hIL11RA (SEQ ID NO: 32) was captured by injecting it over flow cell 2. This created an active target surface on flow cell 2 and a reference surface in flow cell 1, featuring only immobilized Biotin CAPture reagent.

The binding of the polypeptides to the captured target was examined by injecting the analyte over both flow cells, enabling comparative analyses of different compounds' binding to the captured target relative to the reference surface. The compounds of the invention were serially diluted into the running buffer (10 mM HEPES pH 7.4, 150 mM NaCl, 0.05% v/v Surfactant P20, 0.1% w/v HSA), injected at a rate of 30 μl min-1 for 240 seconds, and allowed to dissociate for 4200 seconds. After each analyte injection cycle, the surface was regenerated by injection of regeneration solution (prepared according to manufacturer). This procedure eliminates the captured target, the immobilized CAPture reagent and any bound polypeptides from the surface, preparing it for subsequent interaction experiments.

Polypeptide binding curves undergo processing, which involves subtraction of reference surface signals. This process allowed for the correction of instrument noise, bulk shift, and drift during sample injections. The polypeptides association and dissociation rate constants, namely ka (association rate) and kd (dissociation rate), were extracted by globally fitting a 1:1 Langmuir model to the data using BIACORE™ Insight Evaluation Software (version 5.0.18.22102). The affinity between compounds of the invention and the target was quantified by the equilibrium dissociation constant (K_{D}), calculated from the determined kinetics by the equation K_{D}=kd/ka. The results are presented in Table 8 below.

TABLE 8
Affinity values

| Chem. | K_{D} (nM) |
|---|---|
| 11 | 0.058 |
| 12 | 0.031 |
| 13 | 0.023 |
| 14 | 0.026 |

Analysis of the data shows that the compounds tested are capable of binding hIL11RA. It was further demonstrated that compounds (having high % sequence identity) bind to hIL11RA with an affinity (K_{D}) of less than 0.1 nM.

### Example 7: Surface Plasmon Resonance (SPR) Characterization of the Interaction Between hIL11RA Inhibitors and IL11RA from Non-Human Species

The purpose of this assay is to demonstrate that hIL11RA inhibitors as disclosed herein bind to IL11RA from non-human species and to determine the binding affinity. The assay was conducted similarly to Example 6, except hIL11RA was replaced with IL11RA from rat, pig, cynomolgus monkey and dog and the dissociation time was set to 1800 seconds. The results are presented in Table 9 below.

TABLE 9
Affinity values

| Chem. | K_{D} (nM, rat) | K_{D} (nM, pig) | K_{D} (nM, monkey) | K_{D} (nM, dog) |
|---|---|---|---|---|
| 11 | 4.18 | 0.332 | 0.080 | 0.145 |
| 12 | 1.95 | 0.156 | 0.050 | 0.076 |
| 13 | 1.41 | 0.185 | 0.039 | 0.090 |
| 14 | 1.67 | 0.222 | 0.045 | 0.096 |

Analysis of the data shows that the compounds (having high % sequence identity) exhibit cross-species reactivity binding towards IL11RA from rat, pig, cynomolgus monkey and dog.

### Example 8: In Silico Assessment of Binding Epitopes on hIL11RA

Alphafold v. 2.3 (www.biorxiv.org/content/10.1101/2021.10.04.463034v2) was used to in silico fold the extracellular domain of hIL11RA with a first (Ddi1-based) and a second (Z domain-based) hIL11RA binding polypeptide, namely Chem. 15 (SEQ ID NO:22) and Chem. 16 (SEQ ID NO: 23), respectively.

For multiple sequence alignment, the reduced database setting was applied and the number of recycles was set to 5. All other model parameters were set to ‘default’.

The identified epitopes determined at 3.5 Å distance between any non-hydrogen atoms in the tested compound and hIL11RA comprise the following residues in hIL11RA (SEQ ID NO:1):
Chem.15: Y125, K150, L154, A156, F187, W188, S189, Q213, Q249, H251, F252, L253, D297, F298, L299 and D300 (Epitope “I”)
Chem.16: P141, R143, L145, P167, W168, P169, L175, V198, N199, P200 and A203 (Epitope “II”)

Taken together with Example 9 below the first and second hIL11RA binding polypeptides bind to two distinct epitopes comprising amino acid residues on hIL11RA as shown above.

### Example 9: SPR Based Assessment of Binding Epitopes on hIL11RA

The purpose of this assay is to demonstrate that IL11RA binding polypeptides as disclosed herein bind to two distinct, non-overlapping epitopes on hIL11RA. Further, one of these epitopes overlaps with the IL11 binding site. Binding studies were executed on a BIACORE™ 8K, or BIACORE™ 8K+ (all from CYTIVA™), instruments designed to quantify molecular interactions in real-time utilizing Surface Plasmon Resonance (SPR). The experiments were conducted at 25° C., while samples were maintained at 25° C. in the sample compartment. The SPR instruments' reported signals, expressed in response units (RU), directly correlate with the mass on the sensor chip surfaces within eight parallel channels, each containing two serial flow cells.

The procedure begun with the non-covalent immobilization of biotinylated, recombinant hIL11RA (SEQ ID NO:32) onto flow cell 2 of a Series S Sensor Chip SA (CYTIVA™) following the manufacturer's guidelines. This created an active target surface on flow cell 2 and a reference surface in flow cell 1.

The competition binding of the polypeptides to the captured target was examined by using a dual injection protocol injecting a first analyte (Injection A) at a concentration of 500 nM immediately followed by a second analyte (Injection B) at a concentration of 1000 nM over both flow cells. The protocol enables the qualitative analyses of different compounds' binding to the captured target relative to the reference surface and in the presence of the first analyte already bound to the target. Selected IL11RA binding polypeptides of the invention were diluted into the running buffer (10 mM HEPES pH 7.4, 150 mM NaCl, 0.05% v/v Surfactant P20). Injection A was conducted at a rate of 30 μL min-1 for 120 seconds, immediately followed by Injection B at a rate of 30 μl min-1 for 60 seconds and allowed to dissociate for 1800 seconds.

Polypeptide binding curves undergo processing, which involves subtraction of reference surface signals. This process allowed for the correction of instrument noise, bulk shift, and drift during sample injections. The binding of the polypeptides under the different conditions was analysed using BIACORE™ Insight Evaluation Software (version 5.0.18.22102).

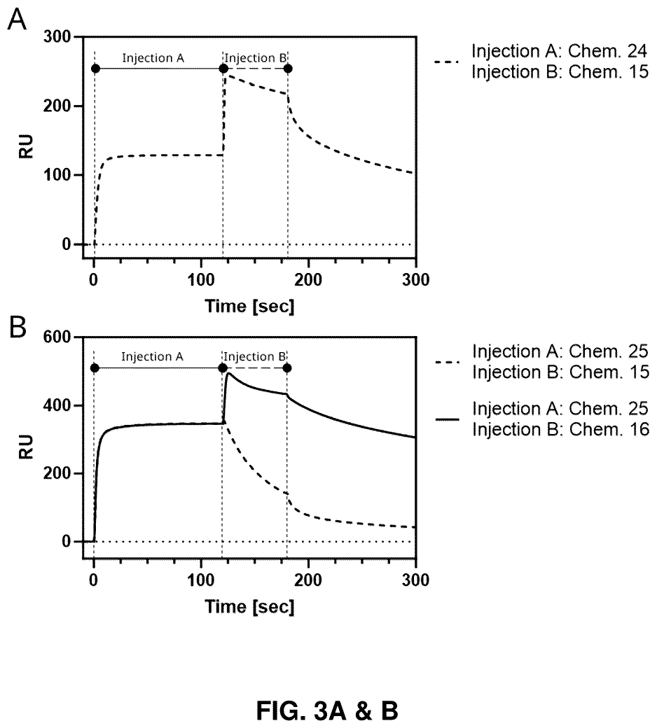

Analysis of the data shows that when hIL11RA is fully saturated with a first polypeptide (Z domain-based) “Chem.24” (SEQ ID NO:33) during “Injection A”, a second polypeptide “Chem. 15” (SEQ ID NO:22) is capable of binding to the receptor simultaneously as evidenced by the increase in measured signal during “Injection B” (FIG. 3A). The increase in measured signal indicates that Chem.24 and Chem. 15 target two distinct, non-overlapping epitopes on hIL11RA and can therefore engage the receptor at the same time.

Furthermore, analysis of the data shows that another polypeptide “Chem. 16” (SEQ ID NO:23) can bind to hIL11RA that is fully saturated by hIL11 “Chem.25” (SEQ ID NO:34) during “Injection A” as evidenced by an increase in signal during “Injection B” (FIG. 3B). This shows that “Chem. 16” binds to an epitope on hIL11RA that does not overlap with the IL11 binding site.

A second polypeptide “Chem. 15” (SEQ ID NO:22) cannot bind to hIL11RA that is fully saturated by hIL11 as evidenced by no measurable increase in signal but rather a decrease during “Injection B” (FIG. 3B).

### Example 10: Screening Substitution Variants of a First Type (Ddi1) of IL11RA Binding Polypeptide

The purpose of this assay is to screen an IL11RA binding polypeptide (Chem.27, SEQ ID NO: 35) for suitable substitutions while retaining or improving binding to IL11RA to serve as potential alternatives at the given positions. Binding studies were executed on a BIACORE™ 8K, or BIACORE™ 8K+ (all from CYTIVA™), instruments designed to quantify molecular interactions in real-time utilizing Surface Plasmon Resonance (SPR). The experiments were conducted at 25° C., while samples were maintained at 25° C. in the sample compartment. The SPR instruments' reported signals, expressed in response units (RU), directly correlate with the mass on the sensor chip surfaces within eight parallel channels, each containing two serial flow cells.

The procedure begun with the non-covalent immobilization of biotinylated, recombinant hIL11RA (Chem.26, SEQ ID NO:32) onto flow cell 2 of a Series S Sensor Chip SA (CYTIVA™) following manufacturer's guidelines. This created an active target surface on flow cell 2 and a reference surface in flow cell 1.

The binding of the polypeptides to the captured target was examined in a screening setup by injecting the analyte over both flow cells, enabling comparative analyses of different compounds' binding to the captured target relative to the reference surface. Each compound was diluted 1:25 into the running buffer (10 mM HEPES pH 7.4, 150 mM NaCl, 0.05% v/v Surfactant P20), injected at a rate of 30 μl min-1 for 60 seconds, and allowed to dissociate for 900 seconds.

Polypeptide binding curves undergo processing, which involves subtraction of reference surface signals. This process allowed for the correction of instrument noise, bulk shift, and drift during sample injections. The binding of the polypeptides was analysed using BIACORE™ Insight Evaluation Software (version 5.0.18.22102). To easily compare compounds from a measurement at one single concentration, custom report points at certain timepoints during the experiment were defined in the Evaluation Software and the respective RU values at each of the report points were extracted. Polypeptides were then ranked according to their % retained binding after 30 seconds of dissociation. The retained binding percentage was calculated by dividing the measured RUs after 30 seconds of dissociation (during the dissociation phase) by the maximally attained signal after 58 seconds of association (during the association phase) and multiplying the result by 100.

% retained binding after 30 s below 20% is considered unattractive. See table 10 below.

TABLE 10
Effect of substitutions

| Substitution | % retained binding after 30 s |
|---|---|
| R7V | 78 |
| R7I | 69 |
| A10L | 37 |
| A10D | 66 |
| A10E | 70 |
| A10T | 75 |
| A10V | 76 |
| A10I | 79 |
| A10Y | 65 |
| W11A | 25 |
| W11R | 68 |
| W11F | 66 |
| Q20A | 42 |
| Q20R | 66 |
| Q20V | 65 |
| Q20L | 67 |
| Q20F | 70 |
| I24A | 43 |
| I24H | 74 |
| I24V | 66 |
| I24L | 75 |
| I24F | 79 |
| I24Y | 80 |
| R28H | 71 |
| R28V | 69 |
| R28L | 67 |
| R28I | 72 |
| R28F | 85 |
| R28Y | 70 |
| R36A | 29 |
| R36H | 75 |
| R36T | 68 |
| R36Q | 66 |
| R36V | 71 |
| R36L | 69 |
| R36I | 70 |
| R36F | 75 |
| R36Y | 77 |
| Wildtype | 73 |
| (Chem. 27) | |

Thus, suitable residues in position 7 are R, V and I. Suitable residues in position 10 are A, D, E, L, T, V, I and Y and so forth.

### Example 11: Screening Substitution Variants of a Second Type (Z Domain-Based) of IL11RA Binding Polypeptide

The purpose of this assay is to screen an IL11RA binding polypeptide (Chem.23, SEQ ID NO: 30) for suitable substitutions while retaining or improving binding to IL11RA to serve as potential alternatives at the given positions. Binding studies were executed on a BIACORE™ 8K, or BIACORE™ 8K+ (all from CYTIVA™), instruments designed to quantify molecular interactions in real-time utilizing Surface Plasmon Resonance (SPR). The experiments were conducted at 25° C., while samples were maintained at 25° C. in the sample compartment. The SPR instruments' reported signals, expressed in response units (RU), directly correlate with the mass on the sensor chip surfaces within eight parallel channels, each containing two serial flow cells.

The procedure begun with the non-covalent immobilization of biotinylated, recombinant hIL11RA (SEQ ID NO:32) onto flow cell 2 of a Series S Sensor Chip SA (CYTIVA™) following the manufacturer's guidelines. This created an active target surface on flow cell 2 and a reference surface in flow cell 1.

The binding of the polypeptides to the captured target was examined in a screening setup by injecting the analyte over both flow cells, enabling comparative analyses of different compounds' binding to the captured target relative to the reference surface. Each compound was diluted 1:25 into the running buffer (10 mM HEPES pH 7.4, 150 mM NaCl, 0.05% v/v Surfactant P20), injected at a rate of 30 μl min-1 for 60 seconds, and allowed to dissociate for 900 seconds.

Polypeptide binding curves undergo processing, which involves subtraction of reference surface signals. This process allowed for the correction of instrument noise, bulk shift, and drift during sample injections. The binding of the polypeptides was analysed using BIACORE™ Insight Evaluation Software (version 5.0.18.22102). To easily compare compounds from a measurement at one single concentration, custom report points at certain timepoints during the experiment were defined in the Evaluation Software and the respective RU values at each of the report points were extracted. Polypeptides were then ranked according to their % retained binding after 30 seconds of dissociation. The retained binding percentage was calculated by dividing the measured RUs after 30 seconds of dissociation (during the dissociation phase) by the maximally attained signal after 58 seconds of association (during the association phase) and multiplying the result by 100. % retained binding after 30 s below 20% is considered unattractive. See table 11 below (noting that position 9 corresponds to the first amino acid residue in the binding motif (BM) of Chem.23 (SEQ ID NO:30)).

TABLE 11
Effect of substitutions

| Substitution | % retained binding after 30 s |
|---|---|
| A9L | 90 |
| Q11A | 96 |
| D14A | 86 |
| D17A | 57 |
| D17T | 82 |
| D17V | 67 |
| D17I | 86 |
| D17L | 81 |
| H18A | 62 |
| H18R | 75 |
| H18S | 73 |
| H18T | 78 |
| H18Q | 78 |
| H18I | 71 |
| H18L | 84 |
| H18F | 89 |
| H18Y | 98 |
| P24A | 89 |
| K27I | 52 |
| L32A | 91 |
| L35A | 58 |
| Wildtype | 98 |
| (Chem. 23) | |

### Example 12: Pharmacokinetics Study in Dog with Chem. 12 and Chem. 13

The aim of the present study was to evaluate the pharmacokinetics of compounds of the invention in male beagle dogs following an intravenous (i.v.) or subcutaneous (s.c) injection. An injection of either Chem. 12 or Chem. 13 was administered to male beagle dogs (n=3 dog per injection, target dose Chem. 12 i.v. 60 nmol/kg, Chem. 12 s.c. 40 nmol/kg and Chem. 13 s.c. nmol/kg).

### In Vivo Procedures

The composition of the injection formulation is listed below:

8 mM phosphate; 250 mM glycerol; pH=7.38-7.45. A single injection of one compound was administered.

Nine male beagle dogs from Marshall BioResources, 69002 Lyon, France ~12 kilos were included in the study. The dogs were single housed during the study in an animal facility with a temperature of 21° C.±3° C. and the light cycle was set to 12:12 hours of light:darkness. Animals had free access to tap water and were given 125 g of TEKLAD DOG DIET™ 2025 C (ENVIGO™ Teklad Diets, Madison, WI, USA) twice daily, with an equal amount of water added, for the duration of the study.

On the day of the compound administration, animals were weighed and received an i.v. or s.c. single bolus injection (T=0 min). Blood samples were collected from a venflon inserted into the cephalica/saphena vein in the front leg (~300 μL blood per sample) in EDTA-coated tubes at the following time points after administration for the measurement of plasma concentrations of test compounds: 5 min, 30 min, 1, 4, 8, 24, 48, 96, 168, 240, 288, 312, 360, 408, 480, 552, and 648 hours. Blood samples were kept on ice until centrifugation (5 min, 6000 rpm at 4° C.) and plasma transferred immediately into Micronic tubes and stored at −20° C. until analyses.

### Bioanalysis

Plasma concentrations of dosed compounds were quantified using the Luminescent Oxygen Channeling Immunoassay (LOCI). Calibrators were prepared by spiking known concentrations of the respective compounds into blank rat or dog plasma at seven different concentrations. Additionally, three control samples were prepared, representing high, medium, and low concentrations.

Unconjugated aldehyde acceptor beads were coupled with an antibody specific to the dosed compounds. A second antibody, also specific to the dosed compounds but with a non-overlapping epitope, was biotinylated. Both antibodies were developed in-house at NOVO NORDISK A/S®.

The assay involved the mixing of conjugated acceptor beads (33.3 μg/mL) and biotinylated antibody (8 nM) in assay buffer (Hepes 25 mM, NaCl 50 mM, EDTA 10 mM, BSA 0.5%, Bovine IgG 0.1%, Dextran T500 2 mg/mL, Tween 20 0.1%, Proclin 300 0.01%, Gentamycin 0.01%, HBR1 0.2 mg/mL, pH 7.4). Plasma samples (1 L) containing either calibrators, control samples, or samples from dosed animals were dispensed into an assay plate (PERKIN ELMER® ALPHAPLATE™ 384).

Subsequently, acceptor beads and biotinylated antibody (15 L) were added to each well of the plate, followed by an incubation period of one hour or overnight, depending on the assay sensitivity. Finally, a solution containing streptavidin-conjugated donor beads (66.7 g/mL) was added to each well. After a 30-minute incubation, the signal was measured using an ENVISION™ reader from PERKIN ELMER®.

A calibration curve was generated by fitting a five-parameter logistic function to the signals from the calibrators, measured in quadruplicate. The assay performance was confirmed by back-calculating the concentrations of each control sample, ensuring they were within 20% of the expected value.

Samples from dosed animals underwent measurement at multiple dilutions to ensure that their concentrations were within the assay range and to ascertain dilution linearity.

### Pharmacokinetic (PK) Analysis

Plasma concentration values of the test compounds were imported into PHOENIX® WINNONLIN® version 8.4 (CERTARA™ L. P. Princeton, NJ, USA) for PK evaluation and non-compartmental analysis (NCA). The area under the plasma concentration-time curves (AUC) were calculated using the “Linear Up Log Down” method. The terminal elimination phase was fitted via linear regression with uniform weighting. Actual doses and nominal sampling time points were used for the NCA calculations. For s.c. administration, the time point for pre-dose samples were set to zero (0 hr) for NCA. Pre-dose time points were excluded from NCA for i.v. administration. Individual concentration values below LLOQ were treated as missing values. The results are shown in the tables below.

TABLE 12
Dog PK results

| Compound | RoA | T½ [hours] |
|---|---|---|
| Chem. 12 | i.v. | 32.1 |
| | | 42.8 |
| | | 25.8 |
| | s.c. | 23.0 |
| | | 93.2 |
| | | 20.6 |
| Chem. 13 | i.v. | 43.8 |
| | | 50.9 |
| | | 47.1 |

RoA: Route of Administration, ADA: Anti-Drug Antibodies, TMDD: Target Mediated Drug Disposition

### Example 13: Pharmacokinetics Study in Rat with Chem. 11-14

The aim of the present study was to evaluate the pharmacokinetics of compounds of the invention in male Sprague Dawley rats following intravenous (i.v.) injection. A single i.v. injection of 4 different compounds (Chem. 11, Chem. 12, Chem. 13, and Chem. 14) was administered to male Sprague Dawley rats (n=3 rats per compound, target dose 1 mg/kg or ~41.5 nmol/kg).

### In Vivo Procedures

The composition of the injection formulation is listed below: 8 mM phosphate; 250 mM glycerol; pH=7.38-7.45.

Twelve male Sprague Dawley rats (from Charles River, Sulzfeld, DE) at ~300 grams were included in the study. The rats were housed together 3 per cage during the study, and kept at a temperature of 20-24° C. and the light cycle was set to 12:12 hours of light:darkness. Animals had free access to tap water and standard Altromin chow diet (Brogården, Lynge, DK) ad libitum for the duration of the study.

On the day of the compound administration, animals were weighed and received an i.v. single bolus injection through the tail vein (T-Omin). Blood samples were collected from the tongue (~150 μL blood per sample) in EDTA-coated tubes at the following time points after administration for the measurement of plasma concentrations of test compounds: 5 min, 1, 4, 24, 48, 72, 96, 120, 144, and 168 hours. Blood samples were kept on ice until centrifugation (5 min, 6000 rpm at 4° C.) and plasma transferred immediately into Micronic tubes and stored at −20° C. until analyses.

### Bioanalysis

Plasma concentrations of dosed compounds were quantified using the Luminescent Oxygen Channeling Immunoassay (LOCI). Calibrators were prepared by spiking known concentrations of the respective compounds into blank rat or dog plasma at seven different concentrations. Additionally, three control samples were prepared, representing high, medium, and low concentrations.

Unconjugated aldehyde acceptor beads were coupled with an antibody specific to the dosed compounds. A second antibody, also specific to the dosed compounds but with a non-overlapping epitope, was biotinylated. Both antibodies were developed in-house at NOVO NORDISK A/S®.

The assay involved the mixing of conjugated acceptor beads (33.3 μg/mL) and biotinylated antibody (8 nM) in assay buffer (Hepes 25 mM, NaCl 50 mM, EDTA 10 mM, BSA 0.5%, Bovine IgG 0.1%, Dextran T500 2 mg/mL, Tween 20 0.1%, Proclin 300 0.01%, Gentamycin 0.01%, HBR1 0.2 mg/mL, pH 7.4). Plasma samples (1 μL) containing either calibrators, control samples, or samples from dosed animals were dispensed into an assay plate (PERKIN ELMER® ALPHAPLATE™ 384).

Subsequently, acceptor beads and biotinylated antibody (15 μL) were added to each well of the plate, followed by an incubation period of one hour or overnight, depending on the assay sensitivity. Finally, a solution containing streptavidin-conjugated donor beads (66.7 μg/mL) was added to each well. After a 30-minute incubation, the signal was measured using an ENVISION™ reader from PERKIN ELMER®.

A calibration curve was generated by fitting a five-parameter logistic function to the signals from the calibrators, measured in quadruplicate. The assay performance was confirmed by back-calculating the concentrations of each control sample, ensuring they were within 20% of the expected value.

Samples from dosed animals underwent measurement at multiple dilutions to ensure that their concentrations were within the assay range and to ascertain dilution linearity.

### Pharmacokinetic (PK) Analysis

Plasma concentration values of the test compounds were imported into PHOENIX® WINNONLIN® version 8.4 (CERTARA™ L. P. Princeton, NJ, USA) for PK evaluation and non-compartmental analysis (NCA). The area under the plasma concentration-time curves (AUC) were calculated using the “Linear Up Log Down” method. The terminal elimination phase was fitted via linear regression with uniform weighting. Actual doses and nominal sampling time points were used for the NCA calculations. Pre-dose time points were excluded from NCA. Individual concentration values below LLOQ were treated as missing values. The results are shown in the table below. In Table 13 (tagged) refers to a variant of the compound including a generic purification tag at the N-terminus of the polypeptide.

TABLE 13
Rat PK results, mean values

| Compound | RoA | n | T½ (hr)* |
|---|---|---|---|
| Chem. 11 (tagged) | i.v. | 3 | 22.2 |
| Chem. 12 (tagged) | i.v. | 2 | 12.6 |
| Chem. 13 (tagged) | i.v. | 3 | 9.68 |
| Chem. 14 (tagged) | i.v. | 3 | 9.44 |
| Chem. 13 | i.v. | 3 | 12.2 |

RoA: Route of Administration,
*Harmonic mean

### Example 14: Pharmacokinetics Study in Humanized IL11RA Mice with Chem. 13

To characterize the pharmacokinetics of Chem. 13, a mouse strain humanized for IL11RA was employed. In these mice (called B-hIL11RA), exon 2-11 of the mouse il11ra was replaced with the human IL11RA sequence on both chromosomes, i.e., the mice express the human IL11RA, while the expression remains under control of the endogenous murine promotor region.

### In Vivo Procedures

Male mice humanized for IL11RA (B-hIL11Ra, Biocytogen, China) were housed in a light, temperature and humidity-controlled environment with free access to standard chow and water. At 8-10 weeks of age, animals were stratified into treatment groups based on body weight. Animals were dosed with a single dose of Chem. 13 (formulated in a suitable vehicle) at a dosing volume of 5 mL/kg via the intravenous (i.v.) (dose: 4, 10 or 20 mg/kg) or the subcutaneous (s.c.) (dose: 20 mg/kg) route of administration (RoA). Blood was collected from n=3 mice per group at each of the following timepoints: 5 and 30 minutes, 1, 2, 4, 6, 8, 12, 20, 24, 28, 40, and 52 hours post-dosing using K2-EDTA as anticoagulant. After separation by centrifugation, plasma was stored at −80° C. until analysis.

### Bioanalysis

Plasma Chem. 13 levels were analyzed using Luminescence Oxygen Channeling Immunoassay (LOCI/AlphaLISA). Two monoclonal antibodies specific to Chem. 13 were conjugated to the acceptor beads and biotinylated, respectively. Both antibodies were developed in-house at NOVO NORDISK A/S®. Calibrators were prepared by spiking compound into mouse plasma at concentrations ranging from 10,000 μM to 7.7 μM. To ensure assay signals fell within the calibration range and to confirm dilution linearity, test samples were diluted 20, 200, and 4000 times in mouse plasma, respectively.

One μL of sample or diluted sample was transferred to a 384-well AlphaPlate (Revvity, USA) and mixed with 15 μL of a mixture of acceptor bead-conjugated antibody and biotinylated antibody diluted in assay buffer. Plates were incubated for 1 hour in the dark at room temperature. Next, 30 μL of streptavidin donor beads diluted in assay buffer were added to each well, and plates were incubated a further 30 minutes. Plates were read on an ENVISION™ plate reader (Revvity, USA) by illuminating donor beads at 680 nm to generate singlet oxygen and detected at 615 nm. Sample concentrations were calculated by fitting a five-parameter logistic (5PL) curve to the calibrator signals and back calculating the sample concentrations. The lower limit of quantification was 70 μM, and the upper limit was 6,000 μM.

### Pharmacokinetic (PK) Analysis

Plasma concentrations of Chem. 13 were imported into PHOENIX® WINNONLIN® version 8.4 (CERTARA™ L.P. Princeton, NJ, USA) for PK evaluation and non-compartmental analysis (NCA). The area under the plasma concentration-time curves (AUC) were calculated using the “Linear Up Log Down” method. The terminal elimination phase was fitted via linear regression with uniform weighting. Nominal doses and sampling time points were used for the NCA calculations. Pre-dose time points were excluded from NCA. Individual concentration values below LLOQ were treated as missing values. The results are shown in table 14 below.

TABEL 14
B-hIL11RA mouse PK results

| Chem. No | RoA | n* | T½ (hours) | Doses (mg/kg) |
|---|---|---|---|---|
| 13 | i.v. | 15 | 15.3 | 20 |
| 13 | i.v. | 15 | 15.2 | 10 |
| 13 | i.v. | 15 | 16.2 | 4 |
| 13 | s.c. | 15 | 14.1 | 20 |

*Sparse sampling calculations

Chem.13 demonstrated linear pharmacokinetics following i.v. administration at 4-20 mg/kg and s.c. dosing at 20 mg/kg. Chem. 13 exhibited an estimated half-life of around 15 hours, supporting its suitability for further development.

### Example 15: Inhibition of IL11RA Signalling by Chem. 13 in Humanized IL11RA Mice

To characterize the inhibition of IL11RA-mediated STAT3 phosphorylation in mouse liver by Chem. 13, a mouse strain humanized for IL11RA was employed. In these mice (called B-hIL11RA), exon 2-11 of the mouse il11ra has been replaced by the human IL11RA sequence on both chromosomes, i.e., the mice express the human IL11RA, while the expression remains under control of the endogenous murine promotor region.

### In Vivo Procedures

Male mice humanized for IL11RA (B-hIL11RA, Biocytogen, China) were housed in a light, temperature and humidity-controlled environment with free access to standard chow and water. At 7-9 weeks of age, animals were stratified into treatment groups based on body weight. All animals were dosed twice, via the intravenous (i.v.) route of administration (RoA): first dose was either vehicle 1 or Chem. 13 (doses: 0.3, 3.0 or 30 mg/kg) followed, 30 minutes later, by a dose of either vehicle 2 or recombinant wild type murine IL11 (rmIL11) (dose: 300 μg/kg). All doses were administered at a volume of 5 ml/kg. 30 min after the second dosing, animals were anesthetized by isoflurane inhalation and subsequently euthanized by cervical dislocation, and liver biopsies were obtained, snap-frozen and stored at −80° C. until analysis.

### Quantification of Liver STAT3 Phosphorylation

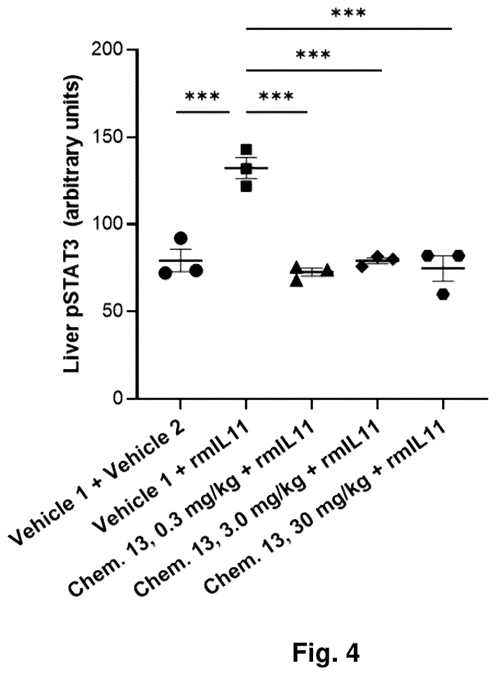

Mouse liver homogenates (50-100 mg tissue) were prepared in 500 μL Surefire lysis buffer containing protease and phosphatase inhibitor (#1861284, PIERCE™), and tissue debris removed by centrifugation (10,000×g, 5 min, 4° C.). Homogenate protein content was determined using a BCA (Bicinchoninic Acid #23227, PIERCE™) assay according to manufacturer's instructions and an equal amount of protein was added from each sample to the assay. Phosphorylation of STAT3 Tyr705 was quantified using the pSTAT3 (Tyr705) AlphaLISA SureFire Ultra Mouse detection kit (#ALSU-PST3-A-HV, Revvity) according to manufacturer's instructions. Statistical analysis was performed using one-way ANOVA in GraphPad Prism and results shown in table below and in FIG. 4.

TABEL 15
Liver pSTAT3 after rmIL11-induction in B-IL11RA mice

| | | | | Liver pSTAT3 |
|---|---|---|---|---|
| | | | | (arbitrary units) |
| Dose 1 | Dose 2 | RoA | n | Mean ± SEM |
| Vehicle 1 | Vehicle 2 | i.v. | 3 | 79.2 ± 6.43*** |
| Vehicle 1 | rmIL11, 300 μg/kg | i.v. | 3 | 132.3 ± 6.06 |
| Chem. 13, 0.3 mg/kg | rmIL11, 300 μg/kg | i.v. | 3 | 72.5 ± 2.29*** |
| Chem. 13, 3.0 mg/kg | rmIL11, 300 μg/kg | i.v. | 3 | 79.1 ± 1.71*** |
| Chem. 13, 30 mg/kg | rmIL11, 300 μg/kg | i.v. | 3 | 74.7 ± 7.33*** |

***p < 0.001: Compared to Vehicle 1/rmIL11 treated B-hIL11RA mice.

Chem. 13 demonstrated target engagement by acute inhibition of rmIL11-induced STAT3 phosphorylation, and thus STAT3 signalling, in the liver of B-hIL11RA mice. A single dose of Chem. 13 prior to rmIL11 administration reduced liver pSTAT3 levels to that of vehicle-induced B-hIL11RA mice demonstrating the IL11RA inhibitory effects of Chem. 13 in a physiological model system.

1.-28. (canceled)

29. An IL11RA inhibitor comprising
a first IL11RA binding polypeptide comprising an amino acid residue sequence selected from

| | |
|---|---|
| i) | A₁S₂A₃T₄F₅P₆X₇Q₈C₉X₁₀X₁₁Q₁₂L₁₃M₁₄D₁₅L₁₆G₁₇F₁₈ |
| | P₁₉X₂₀Y₂₁A₂₂V₂₃X₂₄A₂₅A₂₆L₂₇X₂₈Y₂₉T₃₀N₃₁G₃₂N₃₃C₃₄ |
| | E₃₅X₃₆A₃₇A₃₈S₃₉L₄₀L₄₁F₄₂X₄₃R₄₄ according to SEQ |
| | ID NO: 2 |

wherein, independently of each other,
X₇ is R, V or I;
X₁₀ is A, L, D, E, T, V, I or Y;
X₁₁ is W, A, R or F;
X₂₀ is Q, A, R, V, L or F;
X₂₄ is I, A, H, V, L, F or Y;
X₂₈ is R, H, V, L, I, F or Y;
X₃₆ is R, A, H, T, Q, V, L, I, F or Y;
X₄₃ is A or H;
and
ii) an amino acid residue sequence having at least 90%, at least 91%, at least 92%, at least 93%, at least 94%, at least 95%, at least 96%, at least 97%, at least 98% or at least 99% identity to the sequence defined in i);
and comprising
a second IL11RA binding polypeptide comprising a binding motif (BM), said (BM) consisting of an amino acid residue sequence selected from

| | |
|---|---|
| iii) | XₐWX_{b}AWX_{c}EIX_{d}XₑLPNLNX_{f}WQX_{g}AAFIXₕASLXᵢ |
| | according to SEQ ID NO: 3 |

wherein, independently of each other,
Xₐ is A or L;
X_{b} is Q or A;
X_{c} is D or A;
X_{d} is D, A, T, V, I or L;
Xₑ is H, A, R, S, T, Q, I, L, F or Y;
X_{f} is P or A;
X_{g} is K, A, T or I;
Xₕ is L or A;
Xᵢ is L or A;
and
iv) an amino acid residue sequence which has at least 90%, at least 91%, at least 92%, at least 93%, at least 94%, at least 95%, at least 96%, at least 97%, at least 98% or at least 99% identity to the sequence defined in iii).

30. The IL11RA inhibitor according to claim 29 wherein the second IL11RA binding polypeptide comprises an amino acid residue sequence selected from

| | |
|---|---|
| v) | V₁D₂N₃R₄F₅N₆X₇E₈-(BM)- |
| | X₃₆D₃₇P₃₈S₃₉Q₄₀W₄₁A₄₂N₄₃L₄₄L₄₅X₄₆E₄₇A₄₈R₄₉R₅₀L₅₁ |
| | N₅₂D₅₃A₅₄Q₅₅A₅₆P₅₇X₅₈ according to SEQ ID NO: 4 |

wherein, independently of each other,
X₇ is R or E;
X₃₆ is D or E;
X₄₆ is A or E;
X₅₈ is R or E;
and
vi) an amino acid residue sequence having at least 90%, at least 91%, at least 92%, at least 93%, at least 94%, at least 95%, at least 96%, at least 97%, at least 98% or at least 99% identity to the sequence defined in v).

31. The IL11RA inhibitor according to claim 30 wherein said IL11RA inhibitor comprises
a first IL11RA binding polypeptide selected from the group consisting of:

| | |
|---|---|
| a) | ASATFPRQCAWQLMDLGFPQYAVIAALRYTNGNCERAASLLFAR |
| | according to SEQ ID NO: 5; |

and

| | |
|---|---|
| b) | ASATFPRQCAWQLMDLGFPQYAVIAALRYTNGNCERAASLLFHR |
| | according to SEQ ID NO: 6; |

and
a second IL11RA binding polypeptide selected from the group consisting of:

| | |
|---|---|
| c) | VDNRFNREAWQAWDEIDHLPNLNPWQKAAFILSLLDDPSQWANLL |
| | AEARRLNDAQAPR according to SEQ ID NO: 7; |
| d) | VDNRFNREAWQAWDEIDHLPNLNPWQKAAFILSLLDDPSQWANLL |
| | EEARRLNDAQAPR according to SEQ ID NO: 8; |
| e) | VDNRFNREAWQAWDEIDHLPNLNPWQKAAFILSLLEDPSQWANLL |
| | EEARRLNDAQAPE according to SEQ ID NO: 9; |
| f) | VDNRFNEEAWQAWDEIDHLPNLNPWQKAAFILSLLDDPSQWANLL |
| | EEARRLNDAQAPE according to SEQ ID NO: 10; |

and

| | |
|---|---|
| g) | VDNRFNEEAWQAWDEIDHLPNLNPWQKAAFILSLLEDPSQWANLL |
| | EEARRLNDAQAPE according to SEQ ID NO: 11. |

32. The IL11RA inhibitor according to claim 29 comprising one or more linker(s).

33. The IL11RA inhibitor according to claim 32 wherein said linker(s) is/are GAQPGAQPGAQPGAQP according to SEQ ID NO:13.

34. The IL11RA binding polypeptide or IL11RA inhibitor according to claim 32 further comprising a half-life extending moiety.

35. The IL11RA binding polypeptide or IL11RA inhibitor according to claim 34 wherein the half-life extending moiety comprises the amino acid residue sequence
GEGDLAEAKEAANAELDSYGVSDFYKKLIDKAKTVEGVEALKDAILAALP
according to SEQ ID NO: 12.

36. The IL11RA inhibitor according to claim 29 wherein said IL11RA inhibitor comprises an amino acid residue sequence selected from the group consisting of

| | |
|---|---|
| a) | GEGDLAEAKEAANAELDSYGVSDFYKKLIDKAKTVEGVEALKDAI |
| | LAALPGAQPGAQPGAQPGAQPASATFPRQCAWQLMDLGFPQYAVI |
| | AALRYTNGNCERAASLLFARGAQPGAQPGAQPGAQPVDNRFNREA |
| | WQAWDEIDHLPNLNPWQKAAFILSLLDDPSQWANLLAEARRLNDA |
| | QAPR according to SEQ ID NO: 17 (Chem. 10); |
| b) | GEGDLAEAKEAANAELDSYGVSDFYKKLIDKAKTVEGVEALKDAI |
| | LAALPGAQPGAQPGAQPGAQPASATFPRQCAWQLMDLGFPQYAVI |
| | AALRYTNGNCERAASLLFHRGAQPGAQPGAQPGAQPVDNRFNREA |
| | WQAWDEIDHLPNLNPWQKAAFILSLLDDPSQWANLLEEARRLNDA |
| | QAPR according to SEQ ID NO: 18 (Chem. 11); |
| c) | GEGDLAEAKEAANAELDSYGVSDFYKKLIDKAKTVEGVEALKDAI |
| | LAALPGAQPGAQPGAQPGAQPASATFPRQCAWQLMDLGFPQYAVI |
| | AALRYTNGNCERAASLLFARGAQPGAQPGAQPGAQPVDNRFNREA |
| | WQAWDEIDHLPNLNPWQKAAFILSLLEDPSQWANLLEEARRLNDA |
| | QAPE according to SEQ ID NO: 19 (Chem. 12); |
| d) | GEGDLAEAKEAANAELDSYGVSDFYKKLIDKAKTVEGVEALKDAI |
| | LAALPGAQPGAQPGAQPGAQPASATFPRQCAWQLMDLGFPQYAVI |
| | TAALRYNGNCERAASLLFARGAQPGAQPGAQPGAQPVDNRFNEEA |
| | WQAWDEIDHLPNLNPWQKAAFILSLLDDPSQWANLLEEARRLNDA |
| | QAPE according to SEQ ID NO: 20 (Chem. 13); |

and

| | |
|---|---|
| e) | GEGDLAEAKEAANAELDSYGVSDFYKKLIDKAKTVEGVEALKDAI |
| | LAALPGAQPGAQPGAQPGAQPASATFPRQCAWQLMDLGFPQYAVI |
| | AALRYTNGNCERAASLLFARGAQPGAQPGAQPGAQPVDNRFNEEA |
| | WQAWDEIDHLPNLNPWQKAAFILSLLEDPSQWANLLEEARRLNDA |
| | QAPE according to SEQ ID NO: 21 (Chem. 14). |

37. The hIL11RA inhibitor of claim 36 wherein said inhibitor comprises the amino acid residue sequence
GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFARGAQPGAQPGAQPGAQPVDNRFNREAWQAWDEIDHLPNLNP
WQKAAFILSLLDDPSQWANLLAEARRLNDAQAPR according to
SEQ ID NO: 17 (Chem. 10).

38. The hIL11RA inhibitor of claim 36 wherein said inhibitor comprises the amino acid residue sequence
GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFHRGAQPGAQPGAQPGAQPVDNRFNREAWQAWDEIDHLPNLNP
WQKAAFILSLLDDPSQWANLLEEARRLNDAQAPR according to
SEQ ID NO: 18 (Chem. 11).

39. The hIL11RA inhibitor of claim 36 wherein said inhibitor comprises the amino acid residue sequence
GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFARGAQPGAQPGAQPGAQPVDNRFNREAWQAWDEIDHLPNLNP
WQKAAFILSLLEDPSQWANLLEEARRLNDAQAPE according to
SEQ ID NO: 19 (Chem. 12).

40. The hIL11RA inhibitor of claim 36 wherein said inhibitor comprises the amino acid residue sequence
GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFARGAQPGAQPGAQPGAQPVDNRFNEEAWQAWDEIDHLPNLNP
WQKAAFILSLLDDPSQWANLLEEARRLNDAQAPE according to
SEQ ID NO: 20 (Chem. 13).

41. The hIL11RA inhibitor of claim 36 wherein said inhibitor comprises the amino acid residue sequence
GEGDLAEAKEAANAELDSYGVSDFYKKLIDKAKTVEGVEALKDAILAALP
GAQPGAQPGAQPGAQPASATFPRQCAWQLMDLGFPQYAVIAALRYTNGNC
ERAASLLFARGAQPGAQPGAQPGAQPVDNRFNEEAWQAWDEIDHLPNLNP
WQKAAFILSLLEDPSQWANLLEEARRLNDAQAPE according to
SEQ ID NO: 21 (Chem. 14).

42. The compound of claim 39 comprising an amino acid residue sequence which has at least 90%, at least 91%, at least 92%, at least 93%, at least 94%, at least 95%, at least 96%, at least 97%, at least 98% or at least 99% identity to SEQ ID NO:19.

43. The compound of claim 40 comprising an amino acid residue sequence which has at least 90%, at least 91%, at least 92%, at least 93%, at least 94%, at least 95%, at least 96%, at least 97%, at least 98% or at least 99% identity to SEQ ID NO:20.

44. A pharmaceutical composition comprising the compound according to claim 29, and one or more pharmaceutically acceptable excipients.

45. A pharmaceutical composition comprising the compound according to claim 36, and one or more pharmaceutically acceptable excipients.

46. A pharmaceutical composition comprising the compound according to claim 39, and one or more pharmaceutically acceptable excipients.

47. A pharmaceutical composition comprising the compound according to claim 40, and one or more pharmaceutically acceptable excipients.

48. A method of treating MASH, CVD, a fibro-inflammatory disease or fibrotic disease in a patient in need thereof comprising administering to the patient an effective amount of the compound according to claim 29.

49. A method of treating MASH, CVD, a fibro-inflammatory disease or fibrotic disease in a patient in need thereof comprising administering to the patient an effective amount of the compound according to claim 36.

50. A method of treating MASH, CVD, a fibro-inflammatory disease or fibrotic disease in a patient in need thereof comprising administering to the patient an effective amount of the compound according to claim 39.

51. A method of treating MASH, CVD, a fibro-inflammatory disease or fibrotic disease in a patient in need thereof comprising administering to the patient an effective amount of the compound according to claim 40.

52. A method of treating atrial fibrillation, syncope, edema, tachycardia, or congestive heart failure in a patient in need thereof comprising administering to the patient an effective amount of the compound according to claim 29.

53. A method of treating atrial fibrillation, syncope, edema, tachycardia, or congestive heart failure in a patient in need thereof comprising administering to the patient an effective amount of the compound according to claim 36.

54. A method of treating atrial fibrillation, syncope, edema, tachycardia, or congestive heart failure in a patient in need thereof comprising administering to the patient an effective amount of the compound according to claim 39.

55. A method of treating atrial fibrillation, syncope, edema, tachycardia, or congestive heart failure in a patient in need thereof comprising administering to the patient an effective amount of the compound according to claim 40.


SEQUENCE LISTING (35 sequences)

SEQ ID NO: 1 - protein, 422 aa, synthetic construct
MSSSCSGLSRVLVAVATALVSASSPCPQAWGPPGVQYGQPGRSVKLCCPGVTAGDPVSWFRDGEPKLLQGPDSGLGHELVLAQADSTDEGTYICQTLDGALGGTVTLQLGYPPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGYPRRLRASWTYPASWPCQPHFLLKFRLQYRPAQHPAWSTVEPAGLEEVITDAVAGLPHAVRVSARDFLDAGTWSTWSPEAWGTPSTGTIPKEIPAWGQLHTQPEVEPQVDSPAPPRPSLQPHPRLLDHRDSVEQVAVLASLGILSFLGLVAGALALGLWLRLRRGGKDGSPKPGFLASVIPVDRRPGAPNL

SEQ ID NO: 2 - protein, 44 aa, synthetic construct
  VARIANT 7: note = R, V or I
  VARIANT 10: note = A, L, D, E, T, V, I or Y
  VARIANT 11: note = W, A, R or F
  VARIANT 20: note = Q, A, R, V, L or F
  VARIANT 24: note = I, A, H, V, L, F or Y
  VARIANT 28: note = R, H, V, L, I, F or Y
  VARIANT 36: note = R, A, H, T, Q, V, L, I, F or Y
  VARIANT 43: note = A or H
ASATFPXQCXXQLMDLGFPXYAVXAALXYTNGNCEXAASLLFXR

SEQ ID NO: 3 - protein, 27 aa, synthetic construct
  VARIANT 1: note = A or L
  VARIANT 3: note = Q or A
  VARIANT 6: note = D or A
  VARIANT 9: note = D, A, T, V, I or L
  VARIANT 10: note = P or A
  VARIANT 16: note = P or A
  VARIANT 19: note = K or I
  VARIANT 24: note = L or A
  VARIANT 27: note = L or A
XWXAWXEIXXLPNLNXWQXAAFIXSLX

SEQ ID NO: 4 - protein, 58 aa, Synthetic construct
  VARIANT 7: note = R or E
  VARIANT 9: note = A or L
  VARIANT 11: note = Q or A
  VARIANT 14: note = D or A
  VARIANT 17: note = D, A, T, V, I or L
  VARIANT 18: note = P or A
  VARIANT 24: note = P or A
  VARIANT 27: note = K or I
  VARIANT 32: note = L or A
  VARIANT 35: note = L or A
  VARIANT 36: note = D or E
  VARIANT 46: note = A or E
  VARIANT 58: note = R or E
VDNRFNXEXWXAWXEIXXLPNLNXWQXAAFIXSLXXDPSQWANLLXEARRNLDAQAPX

SEQ ID NO: 5 - protein, 44 aa, Synthetic construct
ASATFPRQCAWQLMDLGFPQYAVIAALRYTNGNCERAASLLFAR

SEQ ID NO: 6 - protein, 44 aa, Synthetic construct
ASATFPRQCAWQLMDLGFPQYAVIAALRYTNGNCERAASLLFHR

SEQ ID NO: 7 - protein, 58 aa, synthetic construct
VDNRFNREAWQAWDEIDHLPNLNPWQKAAFILSLLDDPSQWANLLAEARRLNDAQAPR

SEQ ID NO: 8 - protein, 58 aa, synthetic construct
VDNRFNREAWQAWDEIDHLPNLNPWQKAAFILSLLDDPSQWANLLEEARRLNDAQAPR

SEQ ID NO: 9 - protein, 58 aa, synthetic construct
VDNRFNREAWQAWDEIDHLPNLNPWQKAAFILSLLEDPSQWANLLEEARRLNDAQAPE

SEQ ID NO: 10 - protein, 58 aa, synthetic construct
VDNRFNEEAWQAWDEIDHLPNLNPWQKAAFILSLLDDPSQWANLLEEARRLNDAQAPE

SEQ ID NO: 11 - protein, 58 aa, synthetic construct
VDNRFNEEAWQAWDEIDHLPNLNPWQKAAFILSLLEDPSQWANLLEEARRLNDAQAPE

SEQ ID NO: 12 - protein, 50 aa, synthetic construct
GEGDLAEAKEAANAELDSYGVSDFYKKLIDKAKTVEGVEALKDAILAALP

SEQ ID NO: 13 - protein, 16 aa, synthetic construct
GAQPGAQPGAQPGAQP

SEQ ID NO: 14 - protein, 4 aa, synthetic construct
GAQP

SEQ ID NO: 15 - protein, 4 aa, synthetic construct
GGGS

SEQ ID NO: 16 - protein, 4 aa, synthetic construct
GEQP

SEQ ID NO: 17 - protein, 184 aa, Synthetic construct
GEGDLAEAKEAANAELDSYGVSDFYKKLIDKAKTVEGVEALKDAILAALPGAQPGAQPGAQPGAQPASATFPRQCAWQLMDLGFPQYAVIAALRYTNGNCERAASLLFARGAQPGAQPGAQPGAQPVDNRFNREAWQAWDEIDHLPNLNPWQKAAFILSLLDDPSQWANLLAEARRLNDAQAPR

SEQ ID NO: 18 - protein, 184 aa, Synthetic construct
GEGDLAEAKEAANAELDSYGVSDFYKKLIDKAKTVEGVEALKDAILAALPGAQPGAQPGAQPGAQPASATFPRQCAWQLMDLGFPQYAVIAALRYTNGNCERAASLLFHRGAQPGAQPGAQPGAQPVDNRFNREAWQAWDEIDHLPNLNPWQKAAFILSLLDDPSQWANLLEEARRLNDAQAPR

SEQ ID NO: 19 - protein, 184 aa, Synthetic construct
GEGDLAEAKEAANAELDSYGVSDFYKKLIDKAKTVEGVEALKDAILAALPGAQPGAQPGAQPGAQPASATFPRQCAWQLMDLGFPQYAVIAALRYTNGNCERAASLLFARGAQPGAQPGAQPGAQPVDNRFNREAWQAWDEIDHLPNLNPWQKAAFILSLLEDPSQWANLLEEARRLNDAQAPE

SEQ ID NO: 20 - protein, 184 aa, Synthetic construct
GEGDLAEAKEAANAELDSYGVSDFYKKLIDKAKTVEGVEALKDAILAALPGAQPGAQPGAQPGAQPASATFPRQCAWQLMDLGFPQYAVIAALRYTNGNCERAASLLFARGAQPGAQPGAQPGAQPVDNRFNEEAWQAWDEIDHLPNLNPWQKAAFILSLLDDPSQWANLLEEARRLNDAQAPE

SEQ ID NO: 21 - protein, 184 aa, Synthetic construct
GEGDLAEAKEAANAELDSYGVSDFYKKLIDKAKTVEGVEALKDAILAALPGAQPGAQPGAQPGAQPASATFPRQCAWQLMDLGFPQYAVIAALRYTNGNCERAASLLFARGAQPGAQPGAQPGAQPVDNRFNEEAWQAWDEIDHLPNLNPWQKAAFILSLLEDPSQWANLLEEARRLNDAQAPE

SEQ ID NO: 22 - protein, 45 aa, Synthetic construct
ASATFPRQCAWQLMDLGFPQYAVIAALRYTNGNCERAASLLFKRG

SEQ ID NO: 23 - protein, 58 aa, Synthetic construct; identical to SEQ ID NO: 7

SEQ ID NO: 24 - protein, 137 aa, Synthetic construct
MASATFPRQCAWQLMDLGFPQYAVIAALRYTNGNCERAASLLFKRGEQPGEQPGEQPGEQPGEQPVDNRFNREAWQAWDEIDHLPNLNPWQKAAFILSLLDDPSQWANLLAEARRLNDAQAPRDYKDDDDKHHHHHH

SEQ ID NO: 25 - protein, 137 aa, Synthetic construct
MASATFPRQCAWQLMDLGFPQYAVIAALRYTNGNCERAASLLFKRGAQPGAQPGAQPGAQPGAQPVDNRFNREAWQAWDEIDHLPNLNPWQKAAFILSLLDDPSQWANLLAEARRLNDAQAPRDYKDDDDKHHHHHH

SEQ ID NO: 26 - protein, 133 aa, Synthetic construct
MASATFPRQCAWQLMDLGFPQYAVIAALRYTNGNCERAASLLFKRGEQPGEQPGEQPGEQPVDNRFNREAWQAWDEIDHLPNLNPWQKAAFILSLLDDPSQWANLLAEARRLNDAQAPRDYKDDDDKHHHHHH

SEQ ID NO: 27 - protein, 133 aa, synthetic construct
MASATFPRQCAWQLMDLGFPQYAVIAALRYTNGNCERAASLLFKRGAQPGAQPGAQPGAQPVDNRFNREAWQAWDEIDHLPNLNPWQKAAFILSLLDDPSQWANLLAEARRLNDAQAPRDYKDDDDKHHHHHH

SEQ ID NO: 28 - protein, 70 aa, Synthetic construct
MASATFPRQCAWQLMDLGFPQYAVIAALRYTNGNCERAASLLFKRGGGGGSGGGGSDYKDDDDKHHHHHH

SEQ ID NO: 29 - protein, 84 aa, Synthetic construct
MGEGDRFNREAWQAWDEIDHLPNLNPWQKAAFILSLLDDPSQWANLLEEARRLNDAQAPRGGGGSGGGGSDYKDDDDKHHHHHH

SEQ ID NO: 30 - protein, 83 aa, Synthetic construct
MVDNRFNREAWQAWDEIDHLPNLNPWQKAAFILSLLDDPSQWANLLAEARRLNDAQAPRGGGGSGGGGSDYKDDDDKHHHHHH

SEQ ID NO: 31 - protein, 27 aa, Synthetic construct
AWQAWDEIDHLPNLNPWQKAAFILSLL

SEQ ID NO: 32 - protein, 970 aa, Synthetic construct
HHHHHHGGGSSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGSSGSENLYFQSSSPCPQAWGPPGVQYGQPGRSVKLCCPGVTAGDPVSWFRDGEPKLLQGPDSGLGHELVLAQADSTDEGTYICQTLDGALGGTVTLQLGYPPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGYPRRLRASWTYPASWPCQPHFLLKFRLQYRPAQHPAWSTVEPAGLEEVITDAVAGLPHAVRVSARDFLDAGTWSTWSPEAWGTPSTGTIPKEIPAWGQLHTQPEVEPQVDSPAPPRPSLQPHPRLLDHRDSVGGGGSGLNDIFEAQKIEWHE

SEQ ID NO: 33 - protein, 59 aa, Synthetic construct
GEGDRFNREAWQAWDEIDHLPNLNPWQKAAFILSLLDDPSQWANLLEEARRLNDAQAPR

SEQ ID NO: 34 - protein, 177 aa, synthetic construct
GPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 35 - protein, 70 aa, Synthetic construct
MASATFPRQCAWQLMDLGFPQYAVIAALRYTNGNCERAASLLFARGGGGGSGGGGSDYKDDDDKHHHHHH